SEQUENCE LISTING (18 sequences)

SEQ ID NO: 1 - protein, 485 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 2 - protein, 483 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 3 - protein, 514 aa, Bacillus halmapalus
MKQQKRLYARLLTLLFALIFLLPHSAAAAHHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 4 - protein, 485 aa, Bacillus sp.
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 5 - protein, 483 aa, Bacillus
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 6 - protein, 485 aa, Bacillus
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 7 - protein, 484 aa, Bacillus
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYTFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 8 - protein, 485 aa, Cytophaga
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 9 - protein, 485 aa, Bacillus sp
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 10 - protein, 483 aa, Bacillus sp
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 11 - protein, 485 aa, Bacillus sp
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLACALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 12 - protein, 483 aa, Bacillus licheniformis [sequence omitted]

SEQ ID NO: 13 - protein, 481 aa, Artifical
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 14 - protein, 485 aa, Artifical
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 15 - protein, 485 aa, Bacillus sp
HHNGTNGTMMQYFEWHLPNDGNHWNRLRDDAANLKSKGITAVWIPPAWKGTSQNDVGYGAYDLYDLGEFNQKGTVRTKYGTRSQLQGAVTSLKNNGIQVYGDVVMNHKGGADGTEMVNAVEVNRSNRNQEISGEYTIEAWTKFDFPGRGNTHSNFKWRWYHFDGTDWDQSRQLQNKIYKFRGTGKAWDWEVDIENGNYDYLMYADIDMDHPEVINELRNWGVWYTNTLNLDGFRIDAVKHIKYSYTRDWLTHVRNTTGKPMFAVAEFWKNDLAAIENYLNKTSWNHSVFDVPLHYNLYNASNSGGYFDMRNILNGSVVQKHPIHAVTFVDNHDSQPGEALESFVQSWFKPLAYALILTREQGYPSVFYGDYYGIPTHGVPSMKSKIDPLLQARQTYAYGTQHDYFDHHDIIGWTREGDSSHPNSGLATIMSDGPGGNKWMYVGKHKAGQVWRDITGNRSGTVTINADGWGNFTVNGGAVSVWVKQ

SEQ ID NO: 16 - protein, 480 aa, Bacillus sp
DGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGDVVMNHKMGADFTEAVQAVQVNPTNRWQDISGAYTIDAWTGFDFSGRNNAYSDFKWRWFHFNGVDWDQRYQENHIFRFANTNWNWRVDEENGNYDYLLGSNIDFSHPEVQDELKDWGSWFTDELDLDGYRLDAIKHIPFWYTSDWVRHQRNEADQDLFVVGEYWKDDVGALEFYLDEMNWEMSLFDVPLNYNFYRASQQGGSYDMRNILRGSLVEAHPMHAVTFVDNHDTQPGESLESWVADWFKPLAYATILTREGGYPNVFYGDYYGIPNDNISAKKDMIDELLDARQNYAYGTQHDYFDHWDVVGWTREGSSSRPNSGLATIMSNGPGGSKWMYVGRQNAGQTWTDLTGNNGASVTINGDGWGEFFTNGGSVSVYVNQ

SEQ ID NO: 17 - protein, 483 aa, Bacillus subtilis
HHNGTNGTLMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRANTHSNFKWRWYHFDGVDWDQSRKLNNRIYKFRTKAWDWEVDTEFGNYDYLLYADIDMDHPEVVNELRNWGVWYTNTLGLDGFRIDAVKHIKYSFTRDWINHVRSAIGKNMFAVAEFWKNDLGAIENYLNKTNWNHSVFDVPLHFNLYYASKSGGNYDMRQIFNGTVVQKHPTHAVTFVDNHDSQPEESLESFVREWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

SEQ ID NO: 18 - DNA, 1449 bp, Bacillus
catcataatgggacaaatgggacgatgatgcaatactttgaatggcacttgcctaatgatgggaatcactggaatagattaagagatgatgctagtaatctaagaaatagaggtataaccgctatttggattccgcctgcctggaaagggacttcgcaaaatgatgtggggtatggagcctatgatctttacgatttaggggaatttaatcaaaaggggacggttcgtactaagtatgggacacgtagtcaattggagtctgccatccatgctttaaagaataatggcgttcaagtttatggggatgtagtgatgaaccataaaggaggagctgatgctacagaaaacgttcttgctgtcgaggtgaatccaaataaccggaatcaagaaatatctggggactacacaattgaggcttggactaagtttgattttccagggaggggtaatacatactcagactttaaatggcgttggtatcatttcgatggtgtagattgggatcaatcacgacaattccaaaatcgtatctacaaattccgaggtaaagcttgggattgggaagtagattcggaaaatggaaattatgattatttaatgtatgcagatgtagatatggatcatccggaggtagtaaatgagcttagaagatggggagaatggtatacaaatacattaaatcttgatggatttaggatcgatgcggtgaagcatattaaatatagctttacacgtgattggttgacccatgtaagaaacgcaacgggaaaagaaatgtttgctgttgctgaattttggaaaaatgatttaggtgccttggagaactatttaaataaaacaaactggaatcattctgtctttgatgtcccccttcattataatctttataacgcgtcaaatagtggaggcaactatgacatggcaaaacttcttaatggaacggttgttcaaaagcatccaatgcatgccgtaacttttgtggataatcacgattctcaacctggggaatcattagaatcatttgtacaagaatggtttaagccacttgcttatgcgcttattttaacaagagaacaaggctatccctctgtcttctatggtgactactatggaattccaacacatagtgtcccagcaatgaaagccaagattgatccaatcttagaggcgcgtcaaaattttgcatatggaacacaacatgattattttgaccatcataatataatcggatggacacgtgaaggaaataccacgcatcccaattcaggacttgcgactatcatgtcggatgggccagggggagagaaatggatgtacgtagggcaaaataaagcaggtcaagtttggcatgacataactggaaataaaccaggaacagttacgatcaatgcagatggatgggctaatttttcagtaaatggaggatctgtttccatttgggtgaaacga

(19) United States
(12) Patent Application Publication
(10) Pub. No.: US 2022/0251528 A1
(43) Pub. Date: Aug. 11, 2022

(54) Alpha-Amylase Variants

(71) Applicant: Novozymes A/S, Bagsvaerd (DK)

(72) Inventors: Carsten Andersen, Bagsvaerd (DK); Chakshusmathi Ghadiyaram, Bangalore (IN); Madhupriya Mahankali, Hyderabad (IN); Rajendra Kulothungan Sainathan, Bangalore (IN)

(73) Assignee: Novozymes A/S, Bagsvaerd (DK)

(21) Appl. No.: 17/618,927

(22) PCT Filed: Jun. 23, 2020
(86) PCT No.: PCT/EP2020/067403

§ 371 Date: Dec. 14, 2021

(30) Foreign Application Priority Data
Jun. 24, 2019 (IN) 201941024972

(51) Int. Cl. C12N 9/26; C11D 3/386

(52) U.S. Cl. CPC C12N 9/2414; C11D 3/386; C12Y 302/01001

(57) ABSTRACT

The present invention relates to variants of a parent alpha-amylase having an improved wash performance when compared to the parent alpha-amylase. The present invention also relates to polynucleotides encoding the variants, nucleic acid constructs, vectors, and host cells comprising the polynucleotides, and method of producing the variants of the present invention.

### REFERENCE TO A SEQUENCE LISTING

This application contains a Sequence Listing in computer readable form, which is incorporated herein by reference.

### FIELD OF THE INVENTION

The present invention relates to variants of an alpha-amylase, polynucleotides encoding the variants, and methods of producing the variants.

### BACKGROUND OF THE INVENTION

Alpha-amylases (alpha-1,4-glucan-4-glucanohydrolases, E.C. 3.2.1.1) constitute a group of enzymes, which cat alyses hydrolysis of starch and other linear and branched 1,4-gluosidic oligo- and polysaccharides.

There is a long history of industrial application of alpha amylases in e.g. detergent, baking, brewing, starch liquefaction and saccharification such as in preparation of high fructose syrups or as part of ethanol production from starch. Many of these and other applications of alpha-amylases utilize alpha-amylases derived from microorganisms, in particular bacterial alpha-amylases.

Among the first bacterial alpha-amylases to be used were an alpha-amylase from B. licheniformis, also known as Termamyl, which has been extensively characterized and the crystal structure has been determined for this enzyme. Alkaline amylases, such as the alpha-amylase derived from Bacillus sp. as disclosed in WO 95/26397, form a particular group of alpha-amylases that have found use in detergents. Many of these known bacterial amylases have been modified in order to improve their functionality in a particular application.

Thus, it is an object of the present invention to provide variant polypeptides having alpha-amylase activity that exhibit an improved property, such as specific activity, when compared to the parent polypeptide.

The present invention provides variant polypeptides having alpha-amylase activity and improved property, such as specific activity, compared to its parent polypeptide.

### SUMMARY OF THE INVENTION

The present invention relates to an alpha-amylase variant of a parent alpha-amylase, wherein the variant comprises a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering; and wherein said variant has at least 60%, such as at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent.

The present invention also relates to a polynucleotide encoding a variant according to the invention, a nucleic acid construct comprising the polynucleotide encoding the variant according to the invention, an expression vector comprising the polynucleotide encoding the variant according to the invention, and a host cell comprising the polynucleotide encoding the variant according to the invention.

The present invention also relates to a method of producing an alpha-amylase variant, comprising (a) cultivating the host cell of the invention under conditions suitable for expression of the variant, and (b) recovering the variant.

The present invention further relates to a method of obtaining an alpha-amylase variant of a parent alpha-amylase comprising the steps of: a) introducing a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, b) introducing a deletion at two or more positions in the parent alpha-amylase said positions R181, G182, D183, and G184, using SEQ ID NO: 1 for numbering; said method thereby providing an alpha-amylase variant of said parent alpha-amylase, wherein said variant has at least 60%, such as at least 65%, such as at least 70%, such as at least 75%, such as at least 80%, such as at least 85%, such as at least 90%, such as at least 95%, such as at least 97%, such as at least 99%, but less than 100%, sequence identity to the amino acid sequence to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent.

### Definitions

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

Alpha-Amylases (alpha-1,4-glucan-4-glucanohydrolases, E.C. 3.2.1.1) constitute a group of enzymes, which cat alyze hydrolysis of starch and other linear and branched 1,4-glucosidic oligo- and polysaccharides. In one aspect, the alpha-amylase variant of the present invention have at least 60%, at least 70%, at least 80%, at least 90%, at least 95%, at least 99% but less than 100% of the alpha-amylase activity of the polypeptide of SEQ ID NOs: 1, 2, 3, 4 5, 6, 7, 8, 9, 10, 11, 12, 13, 14, 15, 16 or 17.

Alpha-amylase activity: The term ‘alpha-amylase activity’ as used herein, refers to the activity of an alpha-amylase wherein the activity is determined according to the procedure described in the Examples. The alpha-amylase activity may be determined according to a method using the micro swatch assay which is described in the Example 2.

Amino acid: The term ‘amino acid’ as used herein, refers to the standard twenty genetically-encoded amino acids and their corresponding stereoisomers in the ‘d’ form (as compared to the natural ‘I’ form), omega-amino acids other naturally-occurring amino acids, unconventional amino acids (e.g. α, α -disubstituted amino acids, N-alkyl amino acids, etc.) and chemically derivatised amino acids. Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

When an amino acid is being specifically enumerated, such as ‘alanine’ or ‘Ala’ or ‘A’, the term refers to both 1-alanine and d-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid. In one embodiment, the polypeptides of the invention comprise or consist of 1-amino acids. cDNA: The term “cDNA” as used herein, refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” as used herein, refers to a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” as used herein, refers to nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Corresponding to: The term “corresponding to” as used herein, refers to a way of determining the specific amino acid of a sequence wherein reference is made to a specific amino acid sequence. E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NOs: 1, 2, 3, 4 5, 6, 7, 8, 9, 10, 11, 12, 13, 14, 15, 16 or 17, or any other sequence listed herein, has been described elsewhere herein. Alternative alignment methods may be used and are well-known for the skilled person.

Dish washing composition: The term “dish washing composition” as used herein, refers to all forms of compositions for cleaning hard surfaces. The present invention is not restricted to any particular type of dish wash composition or any particular detergent. Thus, in one embodiment, the dish washing composition is a liquid dish washing composition, a powder dish washing composition, wherein the composition may optionally be in the form of a unit dose.

Enzyme Detergency benefit: The term “enzyme detergency benefit” used herein, refers to the advantageous effect an enzyme may add to a detergent compared to the same detergent without the enzyme. Important detergency benefits which can be provided by enzymes are stain removal with no or very little visible soils after washing and/or cleaning, prevention or reduction of re-deposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to cat alytic stain removal or prevention of re-deposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the cat alytic activity generally is used to cat alyze the formation of bleaching component such as hydrogen peroxide or other peroxides.

Expression: The term “expression” as used herein, refers to any step involved in the production of a variant including, but not limited to, transcription, post-transcriptional modification, translation, post-translational modification, and secretion.

Expression vector: The term “expression vector” as used herein, refers to a linear or circular DNA molecule that comprises a polynucleotide encoding a variant and is operably linked to control sequences that provide for its expression.

Fragment: The term “fragment” as used herein, refers to a polypeptide having one or more (e.g., several) amino acids absent from the amino and/or carboxyl terminus of the mature polypeptide of any one of the parent sequences herein disclosed, such as SEQ ID NOs: SEQ ID NOs: 1, 2, 3, 4, 5, 6, 7, 8, 9, 10, 11, 12, 13, 14, 15, 16 or 17; wherein the fragment has alpha-amylase activity. In one aspect, a fragment contains at least 200 contiguous amino acid residues of SEQ ID NOs: 1, 2, 3, 4, 5, 6, 7, 8, 9, 10, 11, 12, 13, 14, 15, 16 or 17, for example at least 300 contiguous amino acid residues, or at least 350 contiguous amino acid residues, or at least 400 contiguous amino acid residues, or at least 450 contiguous amino acid residues of SEQ ID NOs: 1, 2, 3, 4 5, 6, 7, 8, 9, 10, 11, 12, 13, 14, 15, 16 or 17.

Hard surface cleaning: The term “hard surface cleaning” as used herein, refers to cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics

High Stringency: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” as used herein, refers to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” is defined herein as a characteristic associated with a variant that is improved compared to the parent alpha-amylase. Such improved properties include, but are not limited to, increased amylolytic activity, increased cat alytic efficiency, increased cat alytic rate, increased chemical stability, increased oxidation stability, increased pH activity, increased pH stability, increased specific activity, increased substrate binding, increased substrate cleavage, increased substrate specificity, increased substrate stability, increased surface properties, increased thermal activity, and increased thermostability and increased wash performance such as soil performance e.g. performance to starch containing soils, stain removal, anti-greying, stability e.g. thermostability, pH stability, or stability in the presence of builders, including chelant, stability in powder, liquid or gel detergent formulations or dishwashing compositions, altered temperature-dependent performance and activity profile, pH activity, substrate specificity, product specificity, and chemical stability. The improved property may be any of those herein defined and described, such as increased specific activity. The improved property is increased specific activity in Model A detergent composition. The Improvement Factor (IF) is at least 1.1, at least 1.2, at least 1.3.

Improved Wash Performance: The term “improved wash performance” is defined herein as displaying an alteration of the wash performance of an amylase of the present invention relative to the wash performance of the parent alpha-amylase of SEQ ID NO: 1 or SEQ ID NO: 2. The alteration may e.g. be seen as increased stain removal. The wash performance is improved if the amylase variant of the present invention has improved property relative to said parent polypeptide and said improved property is increased specific activity in Model A detergent composition. The Improvement Factor (IF) is at least 1.1, at least 1.2, at least 1.3.

Isolated: The term “isolated” as used herein, refers to a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Isolated Polynucleotide: The term “isolated polynucleotide” means a polynucleotide that is modified by the hand of man. In one aspect, the isolated polynucleotide is at least 1% pure, e.g., at least 5% pure, at least 10% pure, at least 20% pure, at least 40% pure, at least 60% pure, at least 80% pure, at least 90% pure, and at least 95% pure, as determined by agarose electrophoresis. The polynucleotides may be of genomic, cDNA, RNA, semisynthetic, synthetic origin, or any combinations thereof.

Mature polypeptide: The term “mature polypeptide” as used herein, refers to means a polypeptide in its final form following translation and any post-translational modifications, such as N-terminal processing, C-terminal truncation, glycosylation, phosphorylation, etc. It is known in the art that a host cell may produce a mixture of two of more different mature polypeptides with a different C-terminal and/or N-terminal amino acid) expressed by the same polynucleotide.

Mature polypeptide coding sequence: The term “mature polypeptide coding sequence” as used herein, refers to a polynucleotide that encodes a mature polypeptide having alpha-amylase activity.

Modification: The term “modification”, in the context of the polypeptides of the invention, means that one or more amino acids within the reference amino acid sequence (i.e. SEQ ID NOs: 1, 2, 3, 4 5, 6, 7, 8, 9, 10, 11, 12, 13, 14, 15, 16 or 17) are altered by substitution with a different amino acid, by insertion of an amino acid or by deletion, preferably by at least one deletion. The terms “modification”, “alteration”, and “mutation” may be used interchangeably and constitute the same meaning and purpose.

Medium Stringency: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Mutant: The term “mutant” means a polynucleotide encoding a variant.

Nucleic acid construct: The term “nucleic acid construct” as used herein, refers to a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” as used herein, refers to a configuration in which a control sequence is placed at an appropriate position relative to the coding sequence of a polynucleotide such that the control sequence directs expression of the coding sequence.

Parent or parent alpha-amylase: The term “parent” alpha-amylase as used herein means an alpha-amylase to which modifications are made to produce the variant alpha-amylases of the present invention. This term also refers to the polypeptide with which a variant of the invention is compared. The parent may be a naturally occurring (wild type) polypeptide, or it may even be a variant thereof, prepared by any suitable means. For instance, the parent protein may be a variant of a naturally occurring polypeptide which has been modified or altered in the amino acid sequence. Thus, the parent alpha-amylase may have one or more (or one or several) amino acid substitutions, deletions and/or insertions. Thus, the parent alpha-amylase may be a variant of a parent alpha-amylase. A parent may also be an allelic variant which is a polypeptide encoded by any of two or more alternative forms of a gene occupying the same chromosomal locus. The term “parent” or “parent alpha-amylase” as used herein, refers to the alpha-amylase of SEQ ID NOs: SEQ ID NOs: 1, 2, 3, 4, 5, 6, 7, 8, 9, 10, 11, 12, 13, 14, 15, 16 or 17, or any alpha-amylase having at least 60% sequence identity to any of the polypeptides of SEQ ID NOs: 1, 2, 3, 4, 5, 6, 7, 8, 9, 10, 11, 12, 13, 14, 15, 16 or 17. The parent amylase may also be a polypeptide comprising a fragment of SEQ ID NOs: 1, 2, 3, 4, 5, 6, 7, 8, 9, 10, 11, 12, 13, 14, 15, 16 or 17.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in

Alignment)

Alternatively, the parameters used may be gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment) Subsequence: The term “subsequence” as used herein, refers to a polynucleotide having one or more (e.g., several) nucleotides absent from the 5′ and/or 3′ end of a mature polypeptide coding sequence; wherein the subsequence encodes a fragment having alpha-amylase activity.

Textile: Textile sample CS-27 is obtained from Center For Testmaterials BV, P.O. Box 120, 3133 KT Vlaardingen, the Netherlands.

Textile care benefit: The term “textile care benefits”, as used herein, is defined as not being directly related to cat alytic stain removal or prevention of re-deposition of soils, are also important for enzyme detergency benefits. Examples of such textile care benefits are prevention or reduction of dye transfer from one textile to another textile or another part of the same textile (an effect that is also termed dye transfer inhibition or anti-backstaining), removal of protruding or broken fibers from a textile surface to decrease pilling tendencies or remove already existing pills or fuzz (an effect that also is termed anti-pilling), improvement of the textile-softness, colour clarification of the textile and removal of particulate soils which are trapped in the fibers of the textile. Enzymatic bleaching is a further enzyme detergency benefit where the cat alytic activity generally is used to cat alyze the formation of bleaching component such as hydrogen peroxide or other peroxides or other bleaching species.”

Wild-Type Enzyme: The term “wild-type” alpha-amylase denotes an alpha-amylase expressed by a naturally occurring microorganism, such as a bacterium, yeast or filamentous fungus found in nature. The terms “wild-type enzyme” and “parent enzyme” can be used interchangeably when the parent enzyme is not a variant enzyme.

Variant Enzyme: The terms “variant” or “polypeptide variant” or “polypeptide” or “alpha-amylase variant” when used in relation to a variant of the present invention, as used herein, refer to a polypeptide having alpha-amylase activity comprising an alteration, i.e., a substitution, insertion, and/or deletion, at one or more (e.g., several) positions relative to the ‘parent’ alpha-amylase of SEQ ID NOs: 1 or 2. A substitution means replacement of the amino acid occupying a position with a different amino acid; a deletion means removal of the amino acid occupying a position; and an insertion means adding an amino acid adjacent to and immediately following the amino acid occupying a specific positions of the parent or wild type alpha-amylase. Preferably less than 50 modifications, more preferred less than 30 modifications. The variants of the present invention have at least 20%, e.g., at least 40%, at least 50%, at least 60%, at least 70%, at least 80%, at least 90%, at least 95%, but less than 100% of the alpha-amylase activity of the polypeptide of SEQ ID NOs: 1-17.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “numbering is according to” as used herein, refers to the way each of the amino acid residues in a polypeptide of the present invention is numbered. I.e. the skilled person would know that when, e.g. position 202 is numbered according to SEQ ID NO: 1, he would know that by alignment of any other polypeptide with SEQ ID NO: 1, he will be able to determine the corresponding amino acid residue in the other polypeptide. Alignment of two or more amino acid sequences has been described elsewhere herein.

### Conventions for Designation of Variants

For the purposes of the present invention, the polypeptide disclosed in SEQ ID NO: 1 is used to determine the corresponding amino acid residue in another alpha-amylase polypeptide. Thus, all mentioned positions and specific substitutions refer to the numbering used in SEQ ID NO: 1. However, the skilled person would recognize that the sequence of any other sequence herein disclosed may also be used to determine the corresponding amino acid residue in another alpha-amylase polypeptide. The amino acid sequence of another alpha-amylase is aligned with the mature polypeptide disclosed in SEQ ID NO: 1, and based on the alignment, the amino acid position number corresponding the any amino acid residue in the mature polypeptide disclosed in SEQ IDN 0: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another alpha-amylase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other alpha-amylase has diverged from the polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search dat abases. For example, the PSI-BLAST program generates profiles through an iterative dat abase search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure dat abases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP dat abase. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure dat abases with a structure of interest in order to discover possible structural homologs (e.g., Holm and Park, 2000, Bioinformatics 16: 566-567).

In describing the alpha-amylase variants of the present invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of e.g. threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions: For an amino acid deletion, the following nomenclature is used: Original amino acid, position, *. Accordingly, the deletion of glycine at position 181 is designated as “Ser181*” or “S181*”. Multiple deletions are separated by addition marks (“+”), e.g., “Ser181*+Thr182*” or “S181*+T182*”.

Insertions: For an amino acid insertion, the following nomenclature is used: Original amino acid, position, original amino acid, inserted amino acid. Accordingly the insertion of lysine after e.g. glycine at position 195 is designated “Glyl95GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Glyl95GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations: Variants comprising multiple alterations are separated by addition marks (“+”), e.g., “Arg170Tyr+Glyl95Glu” or “R170Y+G195E” representing a substitution of arginine and glycine at positions 170 and 195 with tyrosine and glutamic acid, respectively.

Different alterations: Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

“Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to an alpha-amylase variant comprising a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering; and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent.

In one aspect, present invention relates to an alpha-amylase variant comprising a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering; and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent.

The present invention provides an alpha-amylase variant of a parent alpha-amylase having alpha-amylase activity, comprising a substitution at one or more (e.g., several) positions corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO:1 for numbering, and the variant has alpha-amylase activity and wherein the alpha-amylase variant has an improved property relative to said parent.

In an embodiment, the variant has sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent alpha-amylase.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 1.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 2.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 3.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 4.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 5.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 6.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 7.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 8.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 9.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 10.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 12.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 13.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 14.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 15.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 16.

In another embodiment, the variant has sequence identity of at least 60%, at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO: 17.

In one aspect, the number of substitutions in the variants of the present invention is 1-20, e.g., 1-10 and 1-5, such as 1, 2, 3, 4, 5, 6, 7, 8, 9 or 10 substitutions.

In another aspect, a variant comprises a substitution at two or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at three or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at four or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at five or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at six or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at seven or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at eight or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at nine or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at ten or more positions corresponding to any of positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, a variant comprises a substitution at each position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 1.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 2.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 3.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 4.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 5.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, TBS, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 6.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 7.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 8.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K4381, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 9.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 10.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 11.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 12.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 13.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 14.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 15.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 16.

In one aspect, a variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V4620, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NO: 17.

In one aspect, the alpha-amylase variant of the present invention have an improved property relative to the parent polypeptide, wherein the improved property is selected from the group consisting of increased cat alytic efficiency, increased cat alytic rate, increased chemical stability, increased oxidation stability, increased pH activity, increased pH stability, increased specific activity, increased stability under storage conditions, increased substrate binding, increased substrate cleavage, increased substrate specificity, increased substrate stability, increased surface properties, increased thermal activity, and increased thermostability.

In one aspect, the alpha-amylase variant of the present invention has improved property relative to said parent polypeptide.

In one aspect, the alpha-amylase variant of the present invention has improved property relative to said parent polypeptide of SEQ ID NO: 1.

In one aspect, the alpha-amylase variant of the present invention has improved property relative to said parent polypeptide of SEQ ID NO: 2.

In one aspect, the alpha-amylase variant of the present invention has improved property relative to said parent polypeptide and wherein said improved property is increased specific activity.

In one aspect, the alpha-amylase variant of the present invention has improved property relative to said parent polypeptide and wherein said increased specific activity in Model A detergent compositon.

In one aspect, the alpha-amylase variant of the present invention has an increased specific activity in Model A detergent compositon measured as Improvement Factor (IF) of >1.0 relative to said parent polypeptide.

In one aspect, the alpha-amylase variant of the present invention has an increased specific activity in Model A detergent compositon measured as Improvement Factor (IF) of ≥1.1 relative to said parent polypeptide.

In one aspect, the alpha-amylase variant of the present invention has an increased specific activity in Model A detergent compositon measured as Improvement Factor (IF) of ≥1.2 relative to said parent polypeptide.

In one aspect, the alpha-amylase variant of the present invention has an increased specific activity in Model A detergent compositon measured as Improvement Factor (IF) of ≥1.3 relative to said parent polypeptide.

In one aspect, the alpha-amylase variant of the present invention has an increased specific activity in Model A detergent compositon measured as Improvement Factor (IF) of ≥1.4 relative to said parent polypeptide.

In one aspect, the alpha-amylase variant of the present invention has an increased specific activity in Model A detergent compositon measured as Improvement Factor (IF) of ≥1.5 relative to said parent polypeptide.

In one aspect, the alpha-amylase variant of the present invention has an increased specific activity in Model A detergent compositon measured as Improvement Factor (IF) of ≥2.0 relative to said parent polypeptide.

In one aspect, the alpha-amylase variant of the present invention has an increased specific activity in Model A detergent compositon measured as Improvement Factor (IF) of ≥2.5 relative to said parent polypeptide.

In another aspect, the variant comprises or consists of a substitution at a position corresponding to position 3. In another aspect, the amino acid at a position corresponding to position 3 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N3A or N3C or N3D or N3E or N3F or N3G or N3H or N3L or N3P or N3Q or N3S or N3T or N3V of the polypeptide of SEQ ID NO: 1.

In another aspect, the variant comprises or consists of a substitution at a position corresponding to position 5. In another aspect, the amino acid at a position corresponding to position 5 is substituted with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of the substitution T5E of the polypeptide of SEQ ID NO: 1.

In another aspect, the variant comprises or consists of a substitution at a position corresponding to position 7. In another aspect, the amino acid at a position corresponding to position 7 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G7E or G7F or G7H or G7K or G7L or G7P or G7R or G7S or G7T or G7V or G7W of the polypeptide of SEQ ID NO: 1.

In another aspect, the variant comprises or consists of a substitution at a position corresponding to position 8. In another aspect, the amino acid at a position corresponding to position 8 is substituted with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of the substitution T8S of the polypeptide of SEQ ID NO: 1.

In another aspect, the variant comprises or consists of a substitution at a position corresponding to position 16. In another aspect, the amino acid at a position corresponding to position 16 is substituted with Ala, Arg, Asn, Asp, Cys, Gln, Glu, Gly, Ile, Leu, Lys, Met, Phe, Pro, Ser, Thr, Trp, Tyr, or Val. In another aspect, the variant comprises or consists of the substitution H16D or H16R of the polypeptide of SEQ ID NO: 1.

In another aspect, the variant comprises or consists of a substitution at a position corresponding to position 18. In another aspect, the amino acid at a position corresponding to position 18 is substituted with Ala, Arg, Asn, Asp, Cys, Gln, Glu, Gly, His, Ile, Leu, Lys, Met, Phe, Ser, Thr, Trp, Tyr, or Val. In another aspect, the variant comprises or consists of the substitution P18D or P18L or P18N of the polypeptide of SEQ ID NO: 1.

In another aspect, the variant comprises or consists of a substitution at a position corresponding to position 25. In another aspect, the amino acid at a position corresponding to position 25 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N25T of the polypeptide of SEQ ID NO: 1.

In another aspect, the variant comprises or consists of a substitution at a position corresponding to position 26. In another aspect, the amino acid at a position corresponding to position 26 is substituted with Ala, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution R26Y of the polypeptide of SEQ ID NO: 1.

In another aspect, the variant comprises or consists of a substitution at a position corresponding to position 29. In another aspect, the amino acid at a position corresponding to position 29 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D29N of the polypeptide of SEQ ID NO: 1.

In another aspect, the variant comprises or consists of a substitution at a position corresponding to position 30. In another aspect, the amino acid at a position corresponding to position 30 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D30F of the polypeptide of SEQ ID NO: 1.

In another aspect, the variant comprises or consists of a substitution at a position corresponding to position 35. In another aspect, the amino acid at a position corresponding to position 35 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R35P of the polypeptide of SEQ ID NO: 1.

In another aspect, the variant comprises or consists of a substitution at a position corresponding to position 37. In another aspect, the amino acid at a position corresponding to position 37 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R37A or R37N of the polypeptide of SEQ ID NO: 1.

In another aspect, the variant comprises or consists of a substitution at a position corresponding to position 40. In another aspect, the amino acid at a position corresponding to position 40 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T40A or T40C or T40D or T40E or T40G or T40H or T40I or T40K or T40N or T40V or T40W of the polypeptide of SEQ ID NO: 1.

In another aspect, the variant comprises or consists of a substitution at a position corresponding to position 41. In another aspect, the amino acid at a position corresponding to position 41 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A40C or A41D or A41E or A41H or A41I or A41K or A41N or A41R or A41T or A41V or A41Y of the polypeptide of SEQ ID NO: 1.

In another aspect, the variant comprises or consists of a substitution at a position corresponding to position 43. In another aspect, the amino acid at a position corresponding to position 43 is substituted with Ala, Arg, Asn, Asp, Cys, Gin, Glu, Gly, His, Ile, Leu, Lys, Met, Phe, Pro, Ser, Thr, Tyr, or Val. In another aspect, the variant comprises or consists of the substitution W43E of the polypeptide of SEQ ID NO: 1.

In another aspect, the variant comprises or consists of a substitution at a position corresponding to position 46. In another aspect, the amino acid at a position corresponding to position 46 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P46A of the polypeptide of SEQ ID NO: 1.

In another aspect, the variant comprises or consists of a substitution at a position corresponding to position 50. In another aspect, the amino acid at a position corresponding to position 50 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G50M or G50P or G50Q of the polypeptide of SEQ ID NO: 1.

In another aspect, the variant comprises or consists of a substitution at a position corresponding to position 57. In another aspect, the amino acid at a position corresponding to position 57 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G57A or G57S of the polypeptide of SEQ ID NO: 1.

In another aspect, the variant comprises or consists of a substitution at a position corresponding to position 60. In another aspect, the amino acid at a position corresponding to position 60 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A60E or A60S or A60V of the polypeptide of SEQ ID NO: 1.

In another aspect, the variant comprises or consists of a substitution at a position corresponding to position 68. In another aspect, the amino acid at a position corresponding to position 68 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E68F or E68Y of the polypeptide of SEQ ID NO: 1.

In another aspect, the variant comprises or consists of a substitution at a position corresponding to position 71. In another aspect, the amino acid at a position corresponding to position 71 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q71F of the polypeptide of SEQ ID NO: 1.

In another aspect, the variant comprises or consists of a substitution at a position corresponding to position 73. In another aspect, the amino acid at a position corresponding to position 73 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G73D or G73H or G73I or G73L or G73Q or G73S or G73T or G73W of the polypeptide of SEQ ID NO: 1.

In another aspect, the variant comprises or consists of a substitution at a position corresponding to position 74. In another aspect, the amino acid at a position corresponding to position 74 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T74F of the polypeptide of SEQ ID NO: 1.

In another aspect, the variant comprises or consists of a substitution at a position corresponding to position 78. In another aspect, the amino acid at a position corresponding to position 78 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K78S or K78T of the polypeptide of SEQ ID NO: 1.

In another aspect, the variant comprises or consists of a substitution at a position corresponding to position 81. In another aspect, the amino acid at a position corresponding to position 81 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T81M of the polypeptide of SEQ ID NO: 1.

In another aspect, the variant comprises or consists of a substitution at a position corresponding to position 84. In another aspect, the amino acid at a position corresponding to position 84 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q84I or Q84W or Q84Y of the polypeptide of SEQ ID NO: 1.

In another aspect, the variant comprises or consists of a substitution at a position corresponding to position 86. In another aspect, the amino acid at a position corresponding to position 86 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E86K or E86S of the polypeptide of SEQ ID NO: 1.

In another aspect, the variant comprises or consists of a substitution at a position corresponding to position 87. In another aspect, the amino acid at a position corresponding to position 87 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S87G or S87L or S87P of the polypeptide of SEQ ID NO: 1.

In another aspect, the variant comprises or consists of a substitution at a position corresponding to position 90. In another aspect, the amino acid at a position corresponding to position 90 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H90P of the polypeptide of SEQ ID NO: 1.

In another aspect, the variant comprises or consists of a substitution at a position corresponding to position 91. In another aspect, the amino acid at a position corresponding to position 91 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A91Q or A91V or A91W of the polypeptide of SEQ ID NO: 1.

In another aspect, the variant comprises or consists of a substitution at a position corresponding to position 93. In another aspect, the amino acid at a position corresponding to position 93 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K93V of the polypeptide of SEQ ID NO: 1.

In another aspect, the variant comprises or consists of a substitution at a position corresponding to position 97. In another aspect, the amino acid at a position corresponding to position 97 is substituted with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of the substitution V97C or V97H of the polypeptide of SEQ ID NO: 1.

In another aspect, the variant comprises or consists of a substitution at a position corresponding to position 98. In another aspect, the amino acid at a position corresponding to position 98 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q98E or Q98F or Q98G or Q98I or Q98K or Q98L or Q98M or Q98R or Q98T or Q98Y of the polypeptide of SEQ ID NO: 1.

In another aspect, the variant comprises or consists of a substitution at a position corresponding to position 103. In another aspect, the amino acid at a position corresponding to position 103 is substituted with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of the substitution V103C or V10G or V103S of the polypeptide of SEQ ID NO: 1.

In another aspect, the variant comprises or consists of a substitution at a position corresponding to position 105. In another aspect, the amino acid at a position corresponding to position 105 is substituted with Ala, Arg, Asn, Asp, Cys, Gln, Glu, Gly, His, Ile, Leu, Lys, Phe, Pro, Ser, Thr, Trp, Tyr, or Val. In another aspect, the variant comprises or consists of the substitution M105F of the polypeptide of SEQ ID NO: 1.

In another aspect, the variant comprises or consists of a substitution at a position corresponding to position 110. In another aspect, the amino acid at a position corresponding to position 110 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G110K or G110R of the polypeptide of SEQ ID NO: 1.

In another aspect, the variant comprises or consists of a substitution at a position corresponding to position 111. In another aspect, the amino acid at a position corresponding to position 111 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A111E or All 1Q of the polypeptide of SEQ ID NO: 1.

In another aspect, the variant comprises or consists of a substitution at a position corresponding to position 113. In another aspect, the amino acid at a position corresponding to position 113 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A113T of the polypeptide of SEQ ID NO: 1.

In another aspect, the variant comprises or consists of a substitution at a position corresponding to position 114. In another aspect, the amino acid at a position corresponding to position 114 is substituted with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of the substitution T114M or T114V of the polypeptide of SEQ ID NO: 1.

In another aspect, the variant comprises or consists of a substitution at a position corresponding to position 116. In another aspect, the amino acid at a position corresponding to position 116 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution N116C or N116Y of the polypeptide of SEQ ID NO: 1.

In another aspect, the variant comprises or consists of a substitution at a position corresponding to position 117. In another aspect, the amino acid at a position corresponding to position 117 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V117F of the polypeptide of SEQ ID NO: 1.

In another aspect, the variant comprises or consists of a substitution at a position corresponding to position 119. In another aspect, the amino acid at a position corresponding to position 119 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A119H or A119L or A119Y of the polypeptide of SEQ ID NO: 1.

In another aspect, the variant comprises or consists of a substitution at a position corresponding to position 120. In another aspect, the amino acid at a position corresponding to position 120 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V120E of the polypeptide of SEQ ID NO: 1.

In another aspect, the variant comprises or consists of a substitution at a position corresponding to position 121. In another aspect, the amino acid at a position corresponding to position 121 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E121I of the polypeptide of SEQ ID NO: 1.

In another aspect, the variant comprises or consists of a substitution at a position corresponding to position 125. In another aspect, the amino acid at a position corresponding to position 125 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N125S of the polypeptide of SEQ ID NO: 1.

In another aspect, the variant comprises or consists of a substitution at a position corresponding to position 126. In another aspect, the amino acid at a position corresponding to position 126 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N1261 of the polypeptide of SEQ ID NO: 1.

In another aspect, the variant comprises or consists of a substitution at a position corresponding to position 128. In another aspect, the amino acid at a position corresponding to position 128 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N128E of the polypeptide of SEQ ID NO: 1.

In another aspect, the variant comprises or consists of a substitution at a position corresponding to position 129. In another aspect, the amino acid at a position corresponding to position 129 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q129T of the polypeptide of SEQ ID NO: 1.

In another aspect, the variant comprises or consists of a substitution at a position corresponding to position 131. In another aspect, the amino acid at a position corresponding to position 131 is substituted with Ala, Arg, Asn, Asp, Cys, Gin, Glu, Gly, His, Leu, Lys, Met, Phe, Pro, Ser, Thr, Trp, Tyr, or Val. In another aspect, the variant comprises or consists of the substitution I131Y of the polypeptide of SEQ ID NO: 1.

In another aspect, the variant comprises or consists of a substitution at a position corresponding to position 132. In another aspect, the amino acid at a position corresponding to position 132 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S132A or S132D or S132H or S132I or S132K or S132L or S132P or S132R or S132T or S132V or S132Y of the polypeptide of SEQ ID NO: 1.

In another aspect, the variant comprises or consists of a substitution at a position corresponding to position 133. In another aspect, the amino acid at a position corresponding to position 133 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G133E or G133L or G133T or G133V of the polypeptide of SEQ ID NO: 1.

In another aspect, the variant comprises or consists of a substitution at a position corresponding to position 135. In another aspect, the amino acid at a position corresponding to position 135 is substituted with Ala, Arg, Asn, Asp, Cys, Gin, Glu, Gly, His, Ile, Leu, Lys, Met, Phe, Pro, Ser, Thr, Trp, or Val. In another aspect, the variant comprises or consists of the substitution Y135C or Y135D or Y135E or Y135P of the polypeptide of SEQ ID NO: 1.

In another aspect, the variant comprises or consists of a substitution at a position corresponding to position 137. In another aspect, the amino acid at a position corresponding to position 137 is substituted with Ala, Arg, Asn, Asp, Cys, Gin, Glu, Gly, His, Leu, Lys, Met, Phe, Pro, Ser, Thr, Trp, Tyr, or Val. In another aspect, the variant comprises or consists of the substitution I137D or I137N or I137Q or I137W or I137Y of the polypeptide of SEQ ID NO: 1.

In another aspect, the variant comprises or consists of a substitution at a position corresponding to position 138. In another aspect, the amino acid at a position corresponding to position 138 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E138S or E138V of the polypeptide of SEQ ID NO: 1.

In another aspect, the variant comprises or consists of a substitution at a position corresponding to position 139. In another aspect, the amino acid at a position corresponding to position 139 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A139L of the polypeptide of SEQ ID NO: 1.

In another aspect, the variant comprises or consists of a substitution at a position corresponding to position 140. In another aspect, the amino acid at a position corresponding to position 140 is substituted with Ala, Arg, Asn, Asp, Cys, Gin, Glu, Gly, His, Ile, Leu, Lys, Met, Phe, Pro, Ser, Thr, Tyr, or Val. In another aspect, the variant comprises or consists of the substitution W140A or W140D or W140M or W140N or W140P or W140S or W140T or W140V of the polypeptide of SEQ ID NO: 1.

In another aspect, the variant comprises or consists of a substitution at a position corresponding to position 141. In another aspect, the amino acid at a position corresponding to position 141 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T141G of the polypeptide of SEQ ID NO: 1.

In another aspect, the variant comprises or consists of a substitution at a position corresponding to position 142. In another aspect, the amino acid at a position corresponding to position 142 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K142E or K142N of the polypeptide of SEQ ID NO: 1.

In another aspect, the variant comprises or consists of a substitution at a position corresponding to position 143. In another aspect, the amino acid at a position corresponding to position 143 is substituted with Ala, Arg, Asn, Asp, Cys, Gin, Glu, Gly, His, Ile, Leu, Lys, Met, Pro, Ser, Thr, Trp, Tyr, or Val. In another aspect, the variant comprises or consists of the substitution F143H or F143I or F143M of the polypeptide of SEQ ID NO: 1.

In another aspect, the variant comprises or consists of a substitution at a position corresponding to position 145. In another aspect, the amino acid at a position corresponding to position 145 is substituted with Ala, Arg, Asn, Asp, Cys, Gin, Glu, Gly, His, Ile, Leu, Lys, Met, Pro, Ser, Thr, Trp, Tyr, or Val. In another aspect, the variant comprises or consists of the substitution F145G or F145H or F145I or F145L or F145S of the polypeptide of SEQ ID NO: 1.

In another aspect, the variant comprises or consists of a substitution at a position corresponding to position 146. In another aspect, the amino acid at a position corresponding to position 146 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P146A or P146H or P146N of the polypeptide of SEQ ID NO: 1.

In another aspect, the variant comprises or consists of a substitution at a position corresponding to position 147. In another aspect, the amino acid at a position corresponding to position 147 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G147D of the polypeptide of SEQ ID NO: 1.

In another aspect, the variant comprises or consists of a substitution at a position corresponding to position 149. In another aspect, the amino acid at a position corresponding to position 149 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G149F or G149K of the polypeptide of SEQ ID NO: 1.

In another aspect, the variant comprises or consists of a substitution at a position corresponding to position 159. In another aspect, the amino acid at a position corresponding to position 159 is substituted with Ala, Arg, Asn, Asp, Cys, Gin, Glu, Gly, His, Ile, Leu, Lys, Met, Phe, Pro, Ser, Thr, Tyr, or Val. In another aspect, the variant comprises or consists of the substitution W159A or W159E of the polypeptide of SEQ ID NO: 1.

In another aspect, the variant comprises or consists of a substitution at a position corresponding to position 161. In another aspect, the amino acid at a position corresponding to position 161 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H161W of the polypeptide of SEQ ID NO: 1.

In another aspect, the variant comprises or consists of a substitution at a position corresponding to position 162. In another aspect, the amino acid at a position corresponding to position 162 is substituted with Ala, Arg, Asn, Asp, Cys, Gin, Glu, Gly, His, Ile, Leu, Lys, Met, Pro, Ser, Thr, Trp, Tyr, or Val. In another aspect, the variant comprises or consists of the substitution F162T of the polypeptide of SEQ ID NO: 1.

In another aspect, the variant comprises or consists of a substitution at a position corresponding to position 165. In another aspect, the amino acid at a position corresponding to position 165 is substituted with Ala, Arg, Asn, Asp, Cys, Gin, Glu, Gly, His, Ile, Leu, Lys, Met, Phe, Pro, Ser, Thr, Trp or Tyr. In another aspect, the variant comprises or consists of the substitution V165P of the polypeptide of SEQ ID NO: 1.

In another aspect, the variant comprises or consists of a substitution at a position corresponding to position 167. In another aspect, the amino acid at a position corresponding to position 167 is substituted with Ala, Arg, Asn, Asp, Cys, Gln, Glu, Gly, His, Ile, Leu, Lys, Met, Phe, Pro, Ser, Thr, Tyr, or Val. In another aspect, the variant comprises or consists of the substitution W167D or W167G or W167P of the polypeptide of SEQ ID NO: 1.

In another aspect, the variant comprises or consists of a substitution at a position corresponding to position 169. In another aspect, the amino acid at a position corresponding to position 169 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q169L or Q169P or Q169V or Q169Y of the polypeptide of SEQ ID NO: 1.

In another aspect, the variant comprises or consists of a substitution at a position corresponding to position 171. In another aspect, the amino acid at a position corresponding to position 171 is substituted with Ala, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution R171P of the polypeptide of SEQ ID NO: 1.

In another aspect, the variant comprises or consists of a substitution at a position corresponding to position 176. In another aspect, the amino acid at a position corresponding to position 176 is substituted with Ala, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution R176E or R176Q or R176V or R176Y of the polypeptide of SEQ ID NO: 1.

In another aspect, the variant comprises or consists of a substitution at a position corresponding to position 177. In another aspect, the amino acid at a position corresponding to position 177 is substituted with Ala, Arg, Asn, Asp, Cys, Gln, Glu, Gly, His, Leu, Lys, Met, Phe, Pro, Ser, Thr, Trp, Tyr, or Val. In another aspect, the variant comprises or consists of the substitution I177H or I177P of the polypeptide of SEQ ID NO: 1.

In another aspect, the variant comprises or consists of a substitution at a position corresponding to position 179. In another aspect, the amino acid at a position corresponding to position 179 is substituted with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of the substitution K179A or K179M or K179V of the polypeptide of SEQ ID NO: 1.

In another aspect, the variant comprises or consists of a substitution at a position corresponding to position 180. In another aspect, the amino acid at a position corresponding to position 180 is substituted with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of the substitution F180D or F180G of the polypeptide of SEQ ID NO: 1.

In another aspect, the variant comprises or consists of a deletion or substitution at a position corresponding to position 181. In another aspect, the amino acid at a position corresponding to position 181 is deleted of the polypeptide of SEQ ID NO: 1. In another aspect, the variant comprises or consists of deletion R181* of the polypeptide of SEQ ID NO: 1. In another aspect, the amino acid at a position corresponding to position 181 is substituted with Ala, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution R181D or R181E or R181G or R181M or R181Y of the polypeptide of SEQ ID NO: 1.

In another aspect, the variant comprises or consists of a substitution at a position corresponding to position 185. In another aspect, the amino acid at a position corresponding to position 185 is substituted with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of the substitution K185T or K185V or K185W of the polypeptide of SEQ ID NO: 1.

In another aspect, the variant comprises or consists of a substitution at a position corresponding to position 186. In another aspect, the amino acid at a position corresponding to position 186 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A186D of the polypeptide of SEQ ID NO: 1.

In another aspect, the variant comprises or consists of a substitution at a position corresponding to position 187. In another aspect, the amino acid at a position corresponding to position 187 is substituted with Ala, Arg, Asn, Asp, Cys, Gln, Glu, Gly, His, Ile, Leu, Lys, Met, Phe, Pro, Ser, Thr, Tyr, or Val. In another aspect, the variant comprises or consists of the substitution W187T of the polypeptide of SEQ ID NO: 1.

In another aspect, the variant comprises or consists of a substitution at a position corresponding to position 195. In another aspect, the amino acid at a position corresponding to position 195 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N195Y of the polypeptide of SEQ ID NO: 1.

In another aspect, the variant comprises or consists of a substitution at a position corresponding to position 196. In another aspect, the amino acid at a position corresponding to position 196 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G196D of the polypeptide of SEQ ID NO: 1.

In another aspect, the variant comprises or consists of a substitution at a position corresponding to position 197. In another aspect, the amino acid at a position corresponding to position 197 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N197V or N197Y of the polypeptide of SEQ ID NO: 1.

In another aspect, the variant comprises or consists of a substitution at a position corresponding to position 204. In another aspect, the amino acid at a position corresponding to position 204 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A204F or A204H or A204L of the polypeptide of SEQ ID NO: 1.

In another aspect, the variant comprises or consists of a substitution at a position corresponding to position 206. In another aspect, the amino acid at a position corresponding to position 206 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V206N of the polypeptide of SEQ ID NO: 1.

In another aspect, the variant comprises or consists of a substitution at a position corresponding to position 208. In another aspect, the amino acid at a position corresponding to position 208 is substituted with Ala, Arg, Asn, Asp, Cys, Gin, Glu, Gly, His, Ile, Leu, Lys, Phe, Pro, Ser, Thr, Trp, Tyr, or Val. In another aspect, the variant comprises or consists of the substitution M208D of the polypeptide of SEQ ID NO: 1.

In another aspect, the variant comprises or consists of a substitution at a position corresponding to position 211. In another aspect, the amino acid at a position corresponding to position 211 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P211C or P211M of the polypeptide of SEQ ID NO: 1.

In another aspect, the variant comprises or consists of a substitution at a position corresponding to position 216. In another aspect, the amino acid at a position corresponding to position 216 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E216L of the polypeptide of SEQ ID NO: 1.

In another aspect, the variant comprises or consists of a substitution at a position corresponding to position 218. In another aspect, the amino acid at a position corresponding to position 218 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R218Q of the polypeptide of SEQ ID NO: 1.

In another aspect, the variant comprises or consists of a substitution at a position corresponding to position 219. In another aspect, the amino acid at a position corresponding to position 219 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R219V of the polypeptide of SEQ ID NO: 1.

In another aspect, the variant comprises or consists of a substitution at a position corresponding to position 225. In another aspect, the amino acid at a position corresponding to position 225 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T225D or T225F or T225H or T225K or T225L or T225N or T225R or T225Y of the polypeptide of SEQ ID NO: 1.

In another aspect, the variant comprises or consists of a substitution at a position corresponding to position 228. In another aspect, the amino acid at a position corresponding to position 228 is substituted with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of the substitution L228V of the polypeptide of SEQ ID NO: 1.

In another aspect, the variant comprises or consists of a substitution at a position corresponding to position 230. In another aspect, the amino acid at a position corresponding to position 230 is substituted with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of the substitution L230C or L230F of the polypeptide of SEQ ID NO: 1.

In another aspect, the variant comprises or consists of a substitution at a position corresponding to position 231. In another aspect, the amino acid at a position corresponding to position 231 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D213G or D231T or D231V of the polypeptide of SEQ ID NO: 1.

In another aspect, the variant comprises or consists of a substitution at a position corresponding to position 235. In another aspect, the amino acid at a position corresponding to position 235 is substituted with Ala, Arg, Asn, Asp, Cys, Gln, Glu, Gly, His, Leu, Lys, Met, Phe, Pro, Ser, Thr, Trp, Tyr, or Val. In another aspect, the variant comprises or consists of the substitution I235A of the polypeptide of SEQ ID NO: 1.

In another aspect, the variant comprises or consists of a substitution at a position corresponding to position 242. In another aspect, the amino acid at a position corresponding to position 242 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K242L of the polypeptide of SEQ ID NO: 1.

In another aspect, the variant comprises or consists of a substitution at a position corresponding to position 243. In another aspect, the amino acid at a position corresponding to position 243 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution Y243D or Y243M or Y243N of the polypeptide of SEQ ID NO: 1.

In another aspect, the variant comprises or consists of a substitution at a position corresponding to position 247. In another aspect, the amino acid at a position corresponding to position 247 is substituted with Ala, Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R247G or R247P or R247S of the polypeptide of SEQ ID NO: 1.

In another aspect, the variant comprises or consists of a substitution at a position corresponding to position 250. In another aspect, the amino acid at a position corresponding to position 250 is substituted with Ala, Arg, Asn, Asp, Cys, Gin, Glu, Gly, His, Ile, Lys, Met, Phe, Pro, Ser, Thr, Trp, Tyr, or Val. In another aspect, the variant comprises or consists of the substitution L250S or L250Y of the polypeptide of SEQ ID NO: 1.

In another aspect, the variant comprises or consists of a substitution at a position corresponding to position 251. In another aspect, the amino acid at a position corresponding to position 251 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T251D of the polypeptide of SEQ ID NO: 1.

In another aspect, the variant comprises or consists of a substitution at a position corresponding to position 252. In another aspect, the amino acid at a position corresponding to position 252 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H252P of the polypeptide of SEQ ID NO: 1.

In another aspect, the variant comprises or consists of a substitution at a position corresponding to position 253. In another aspect, the amino acid at a position corresponding to position 253 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V253S or V253F of the polypeptide of SEQ ID NO: 1.

In another aspect, the variant comprises or consists of a substitution at a position corresponding to position 254. In another aspect, the amino acid at a position corresponding to position 254 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R254D or R254F or R254H or R254P or R254T or R254V or R254W of the polypeptide of SEQ ID NO: 1.

In another aspect, the variant comprises or consists of a substitution at a position corresponding to position 255. In another aspect, the amino acid at a position corresponding to position 255 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N255C or N255F or N255P of the polypeptide of SEQ ID NO: 1.

In another aspect, the variant comprises or consists of a substitution at a position corresponding to position 257. In another aspect, the amino acid at a position corresponding to position 257 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T257A or T257C or T257D or T257E or T257G or T257P or T257Q or T257S or T257W or T257Y of the polypeptide of SEQ ID NO: 1.

In another aspect, the variant comprises or consists of a substitution at a position corresponding to position 259. In another aspect, the amino acid at a position corresponding to position 259 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K259P or K259V or K259W or K259Y of the polypeptide of SEQ ID NO: 1.

In another aspect, the variant comprises or consists of a substitution at a position corresponding to position 260. In another aspect, the amino acid at a position corresponding to position 260 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E260V of the polypeptide of SEQ ID NO: 1.

In another aspect, the variant comprises or consists of a substitution at a position corresponding to position 261. In another aspect, the amino acid at a position corresponding to position 261 is substituted with Ala, Arg, Asn, Asp, Cys, Gin, Glu, Gly, His, Ile, Leu, Lys, Phe, Pro, Ser, Thr, Trp, Tyr, or Val. In another aspect, the variant comprises or consists of the substitution M261D or M261F or M261P or M261R or M261S or M261W of the polypeptide of SEQ ID NO: 1.

In another aspect, the variant comprises or consists of a substitution at a position corresponding to position 262. In another aspect, the amino acid at a position corresponding to position 262 is substituted with Ala, Arg, Asn, Asp, Cys, Gin, Glu, Gly, His, Ile, Leu, Lys, Met, Pro, Ser, Thr, Trp, Tyr, or Val. In another aspect, the variant comprises or consists of the substitution F262A or F262C or F262E or F262H or F262I or F262K or F262L or F262N or F262P or F262Q or F262R or F262S or F262T or F262V or F262W of the polypeptide of SEQ ID NO: 1.

In another aspect, the variant comprises or consists of a substitution at a position corresponding to position 263. In another aspect, the amino acid at a position corresponding to position 263 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A263E or A263H or A263W of the polypeptide of SEQ ID NO: 1.

In another aspect, the variant comprises or consists of a substitution at a position corresponding to position 265. In another aspect, the amino acid at a position corresponding to position 265 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A265Q of the polypeptide of SEQ ID NO: 1.

In another aspect, the variant comprises or consists of a substitution at a position corresponding to position 272. In another aspect, the amino acid at a position corresponding to position 272 is substituted with Ala, Arg, Asn, Asp, Cys, Gin, Glu, Gly, His, Ile, Lys, Met, Phe, Pro, Ser, Thr, Trp, Tyr, or Val. In another aspect, the variant comprises or consists of the substitution L272W of the polypeptide of SEQ ID NO: 1.

In another aspect, the variant comprises or consists of a substitution at a position corresponding to position 273. In another aspect, the amino acid at a position corresponding to position 273 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G273Q of the polypeptide of SEQ ID NO: 1.

In another aspect, the variant comprises or consists of a substitution at a position corresponding to position 274. In another aspect, the amino acid at a position corresponding to position 274 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A274D of the polypeptide of SEQ ID NO: 1.

In another aspect, the variant comprises or consists of a substitution at a position corresponding to position 279. In another aspect, the amino acid at a position corresponding to position 279 is substituted with Ala, Arg, Asn, Asp, Cys, Gin, Glu, Gly, His, Ile, Lys, Met, Phe, Pro, Ser, Thr, Trp, Tyr, or Val. In another aspect, the variant comprises or consists of the substitution L279I of the polypeptide of SEQ ID NO: 1.

In another aspect, the variant comprises or consists of a substitution at a position corresponding to position 280. In another aspect, the amino acid at a position corresponding to position 280 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N280P of the polypeptide of SEQ ID NO: 1.

In another aspect, the variant comprises or consists of a substitution at a position corresponding to position 281. In another aspect, the amino acid at a position corresponding to position 281 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K281H or K281I or K281S of the polypeptide of SEQ ID NO: 1.

In another aspect, the variant comprises or consists of a substitution at a position corresponding to position 283. In another aspect, the amino acid at a position corresponding to position 283 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N283P of the polypeptide of SEQ ID NO: 1.

In another aspect, the variant comprises or consists of a substitution at a position corresponding to position 284. In another aspect, the amino acid at a position corresponding to position 284 is substituted with Ala, Arg, Asn, Asp, Cys, Gin, Glu, Gly, His, Ile, Leu, Lys, Met, Phe, Pro, Ser, Thr, Tyr, or Val. In another aspect, the variant comprises or consists of the substitution W284A or W284D or W284E or W284G or W284I or W284K or W284M or W284N or W284P or W284Q or W284S or W284T or W284V or W284Y of the polypeptide of SEQ ID NO: 1.

In another aspect, the variant comprises or consists of a substitution at a position corresponding to position 287. In another aspect, the amino acid at a position corresponding to position 287 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S287I or S287K or S287L or S287P or S287R or S287T or S287V or S287Y of the polypeptide of SEQ ID NO: 1.

In another aspect, the variant comprises or consists of a substitution at a position corresponding to position 292. In another aspect, the amino acid at a position corresponding to position 292 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P292L or P292M or P292Y of the polypeptide of SEQ ID NO: 1.

In another aspect, the variant comprises or consists of a substitution at a position corresponding to position 294. In another aspect, the amino acid at a position corresponding to position 294 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H294N or H249S of the polypeptide of SEQ ID NO: 1.

In another aspect, the variant comprises or consists of a substitution at a position corresponding to position 296. In another aspect, the amino acid at a position corresponding to position 296 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N296T of the polypeptide of SEQ ID NO: 1.

In another aspect, the variant comprises or consists of a substitution at a position corresponding to position 298. In another aspect, the amino acid at a position corresponding to position 298 is substituted with Ala, Arg, Asn, Asp, Cys, Gin, Glu, Gly, His, Ile, Leu, Lys, Met, Phe, Pro, Ser, Thr, Trp, or Val. In another aspect, the variant comprises or consists of the substitution Y298D or Y298E of the polypeptide of SEQ ID NO: 1.

In another aspect, the variant comprises or consists of a substitution at a position corresponding to position 299. In another aspect, the amino acid at a position corresponding to position 299 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N299T of the polypeptide of SEQ ID NO: 1.

In another aspect, the variant comprises or consists of a substitution at a position corresponding to position 300. In another aspect, the amino acid at a position corresponding to position 300 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A300G of the polypeptide of SEQ ID NO: 1.

In another aspect, the variant comprises or consists of a substitution at a position corresponding to position 303. In another aspect, the amino acid at a position corresponding to position 303 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S303A or S303C or S303D or S303E or S303F or S303G or S303H or S303L or S303M or S303N or S303Q or S303T or S303V or S303Y of the polypeptide of SEQ ID NO: 1.

In another aspect, the variant comprises or consists of a substitution at a position corresponding to position 305. In another aspect, the amino acid at a position corresponding to position 305 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G305E of the polypeptide of SEQ ID NO: 1.

In another aspect, the variant comprises or consists of a substitution at a position corresponding to position 307. In another aspect, the amino acid at a position corresponding to position 307 is substituted with Ala, Arg, Asn, Asp, Cys, Gin, Glu, Gly, His, Ile, Leu, Lys, Met, Phe, Pro, Ser, Thr, Trp, or Val. In another aspect, the variant comprises or consists of the substitution Y307S of the polypeptide of SEQ ID NO: 1.

In another aspect, the variant comprises or consists of a substitution at a position corresponding to position 310. In another aspect, the amino acid at a position corresponding to position 310 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A310D or A310N of the polypeptide of SEQ ID NO: 1.

In another aspect, the variant comprises or consists of a substitution at a position corresponding to position 311. In another aspect, the amino acid at a position corresponding to position 311 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K311C or K311C of the polypeptide of SEQ ID NO: 1.

In another aspect, the variant comprises or consists of a substitution at a position corresponding to position 313. In another aspect, the amino acid at a position corresponding to position 313 is substituted with Ala, Arg, Asn, Asp, Cys, Gin, Glu, Gly, His, Ile, Lys, Met, Phe, Pro, Ser, Thr, Trp, Tyr, or Val. In another aspect, the variant comprises or consists of the substitution L313Q of the polypeptide of SEQ ID NO: 1.

In another aspect, the variant comprises or consists of a substitution at a position corresponding to position 314. In another aspect, the amino acid at a position corresponding to position 314 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N314S or N314P of the polypeptide of SEQ ID NO: 1.

In another aspect, the variant comprises or consists of a substitution at a position corresponding to position 315. In another aspect, the amino acid at a position corresponding to position 315 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G315E or G315M or G315V of the polypeptide of SEQ ID NO: 1.

In another aspect, the variant comprises or consists of a substitution at a position corresponding to position 319. In another aspect, the amino acid at a position corresponding to position 319 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q319C or Q319E of the polypeptide of SEQ ID NO: 1.

In another aspect, the variant comprises or consists of a substitution at a position corresponding to position 320. In another aspect, the amino acid at a position corresponding to position 320 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K320S or K320W of the polypeptide of SEQ ID NO: 1.

In another aspect, the variant comprises or consists of a substitution at a position corresponding to position 321. In another aspect, the amino acid at a position corresponding to position 321 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H321F or H321K or H321L or H321R or H321T or H321V or H321W of the polypeptide of SEQ ID NO: 1.

In another aspect, the variant comprises or consists of a substitution at a position corresponding to position 322. In another aspect, the amino acid at a position corresponding to position 322 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P322C or P322E or P322H or P322T or P322Y of the polypeptide of SEQ ID NO: 1.

In another aspect, the variant comprises or consists of a substitution at a position corresponding to position 323. In another aspect, the amino acid at a position corresponding to position 323 is substituted with Ala, Arg, Asn, Asp, Cys, Gin, Glu, Gly, His, Ile, Leu, Lys, Phe, Pro, Ser, Thr, Trp, Tyr, or Val. In another aspect, the variant comprises or consists of the substitution M323A or M323C or M323D or M323E or M323Gor M323H or M323L or M323N or M323P or M323R or M323S or M323T or M323W or M323Y of the polypeptide of SEQ ID NO: 1.

In another aspect, the variant comprises or consists of a substitution at a position corresponding to position 324. In another aspect, the amino acid at a position corresponding to position 324 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H324A or H3241 or H324P or H324T or H324W or H324Y of the polypeptide of SEQ ID NO: 1.

In another aspect, the variant comprises or consists of a substitution at a position corresponding to position 334. In another aspect, the amino acid at a position corresponding to position 334 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S334V of the polypeptide of SEQ ID NO: 1.

In another aspect, the variant comprises or consists of a substitution at a position corresponding to position 337. In another aspect, the amino acid at a position corresponding to position 337 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G337V of the polypeptide of SEQ ID NO: 1.

In another aspect, the variant comprises or consists of a substitution at a position corresponding to position 340. In another aspect, the amino acid at a position corresponding to position 340 is substituted with Ala, Arg, Asn, Asp, Cys, Gin, Glu, Gly, His, Ile, Lys, Met, Phe, Pro, Ser, Thr, Trp, Tyr, or Val. In another aspect, the variant comprises or consists of the substitution L340M of the polypeptide of SEQ ID NO: 1.

In another aspect, the variant comprises or consists of a substitution at a position corresponding to position 342. In another aspect, the amino acid at a position corresponding to position 342 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S342A or S342G or S342T of the polypeptide of SEQ ID NO: 1.

In another aspect, the variant comprises or consists of a substitution at a position corresponding to position 344. In another aspect, the amino acid at a position corresponding to position 344 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V344A or V344C or V3441 of the polypeptide of SEQ ID NO: 1.

In another aspect, the variant comprises or consists of a substitution at a position corresponding to position 345. In another aspect, the amino acid at a position corresponding to position 345 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q345N of the polypeptide of SEQ ID NO: 1.

In another aspect, the variant comprises or consists of a substitution at a position corresponding to position 346. In another aspect, the amino acid at a position corresponding to position 346 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E346A of the polypeptide of SEQ ID NO: 1.

In another aspect, the variant comprises or consists of a substitution at a position corresponding to position 347. In another aspect, the amino acid at a position corresponding to position 347 is substituted with Ala, Arg, Asn, Asp, Cys, Gin, Glu, Gly, His, Ile, Leu, Lys, Met, Phe, Pro, Ser, Thr, Tyr, or Val. In another aspect, the variant comprises or consists of the substitution W347A or W347E or W347F or W347G or W347H or W347K or W347N or W347P or W347R of the polypeptide of SEQ ID NO: 1.

In another aspect, the variant comprises or consists of a substitution at a position corresponding to position 350. In another aspect, the amino acid at a position corresponding to position 350 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P350A or P350C or P350N or P350T of the polypeptide of SEQ ID NO: 1.

In another aspect, the variant comprises or consists of a substitution at a position corresponding to position 359. In another aspect, the amino acid at a position corresponding to position 359 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R359C or R359F or R359G or R359M or R359S or R359W of the polypeptide of SEQ ID NO: 1.

In another aspect, the variant comprises or consists of a substitution at a position corresponding to position 360. In another aspect, the amino acid at a position corresponding to position 360 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E360D or E360H or E360P or E360T or E360W of the polypeptide of SEQ ID NO: 1.

In another aspect, the variant comprises or consists of a substitution at a position corresponding to position 361. In another aspect, the amino acid at a position corresponding to position 361 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q361D or Q361H or Q361I or Q361K or Q361L or Q361M or Q361N or Q361P or Q361R or Q361S or Q361T or Q361V or Q361W or Q361Y of the polypeptide of SEQ ID NO: 1.

In another aspect, the variant comprises or consists of a substitution at a position corresponding to position 362. In another aspect, the amino acid at a position corresponding to position 362 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G326M of the polypeptide of SEQ ID NO: 1.

In another aspect, the variant comprises or consists of a substitution at a position corresponding to position 362. In another aspect, the amino acid at a position corresponding to position 362 is substituted with Ala, Arg, Asn, Asp, Cys, Gin, Glu, Gly, His, Ile, Leu, Lys, Met, Phe, Pro, Ser, Thr, Trp, or Val. In another aspect, the variant comprises or consists of the substitution Y363A or Y363E or Y363F or Y363G or Y3631 or Y363L or Y363Q or Y363R or Y363S or Y363T or Y363V or Y363W of the polypeptide of SEQ ID NO: 1.

In another aspect, the variant comprises or consists of a substitution at a position corresponding to position 364. In another aspect, the amino acid at a position corresponding to position 364 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P364C of the polypeptide of SEQ ID NO: 1.

In another aspect, the variant comprises or consists of a substitution at a position corresponding to position 368. In another aspect, the amino acid at a position corresponding to position 368 is substituted with Ala, Arg, Asn, Asp, Cys, Gin, Glu, Gly, His, Ile, Leu, Lys, Met, Phe, Pro, Ser, Thr, Trp, or Val. In another aspect, the variant comprises or consists of the substitution Y368C or Y368Q or Y368R or Y368T of the polypeptide of SEQ ID NO: 1.

In another aspect, the variant comprises or consists of a substitution at a position corresponding to position 372. In another aspect, the amino acid at a position corresponding to position 372 is substituted with Ala, Arg, Asn, Asp, Cys, Gin, Glu, Gly, His, Ile, Leu, Lys, Met, Phe, Pro, Ser, Thr, Trp, or Val. In another aspect, the variant comprises or consists of the substitution Y372C, Y327G, Y327N, Y327T of the polypeptide of SEQ ID NO: 1.

In another aspect, the variant comprises or consists of a substitution at a position corresponding to position 375. In another aspect, the amino acid at a position corresponding to position 375 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P375Q or P375T of the polypeptide of SEQ ID NO: 1.

In another aspect, the variant comprises or consists of a substitution at a position corresponding to position 380. In another aspect, the amino acid at a position corresponding to position 380 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P380F of the polypeptide of SEQ ID NO: 1.

In another aspect, the variant comprises or consists of a substitution at a position corresponding to position 382. In another aspect, the amino acid at a position corresponding to position 382 is substituted with Ala, Arg, Asn, Asp, Cys, Gin, Glu, Gly, His, Ile, Leu, Lys, Phe, Pro, Ser, Thr, Trp, Tyr, or Val. In another aspect, the variant comprises or consists of the substitution M3281 of the polypeptide of SEQ ID NO: 1.

In another aspect, the variant comprises or consists of a substitution at a position corresponding to position 383. In another aspect, the amino acid at a position corresponding to position 383 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K3830 or K383L or K383P or K383T of the polypeptide of SEQ ID NO: 1.

In another aspect, the variant comprises or consists of a substitution at a position corresponding to position 384. In another aspect, the amino acid at a position corresponding to position 384 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A384D or A384N of the polypeptide of SEQ ID NO: 1.

In another aspect, the variant comprises or consists of a substitution at a position corresponding to position 385. In another aspect, the amino acid at a position corresponding to position 385 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K385N or K385V of the polypeptide of SEQ ID NO: 1.

In another aspect, the variant comprises or consists of a substitution at a position corresponding to position 387. In another aspect, the amino acid at a position corresponding to position 387 is substituted with Ala, Arg, Asn, Cys, Gin, Glu, Gly, His, Ile, Leu, Lys, Met, Phe, Pro, Ser, Thr, Trp, Tyr, or Val. In another aspect, the variant comprises or consists of the substitution D387C or D387P or D387R or D387W of the polypeptide of SEQ ID NO: 1.

In another aspect, the variant comprises or consists of a substitution at a position corresponding to position 388. In another aspect, the amino acid at a position corresponding to position 388 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P388I of the polypeptide of SEQ ID NO: 1.

In another aspect, the variant comprises or consists of a substitution at a position corresponding to position 391. In another aspect, the amino acid at a position corresponding to position 391 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E399M of the polypeptide of SEQ ID NO: 1.

In another aspect, the variant comprises or consists of a substitution at a position corresponding to position 393. In another aspect, the amino acid at a position corresponding to position 393 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R393K or R393M of the polypeptide of SEQ ID NO: 1.

In another aspect, the variant comprises or consists of a substitution at a position corresponding to position 395. In another aspect, the amino acid at a position corresponding to position 395 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N395C or N395D or N395K or N395V or N395W of the polypeptide of SEQ ID NO: 1.

In another aspect, the variant comprises or consists of a substitution at a position corresponding to position 396. In another aspect, the amino acid at a position corresponding to position 396 is substituted with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of the substitution F396E or F396G or F396T of the polypeptide of SEQ ID NO: 1.

In another aspect, the variant comprises or consists of a substitution at a position corresponding to position 397. In another aspect, the amino acid at a position corresponding to position 397 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A379V of the polypeptide of SEQ ID NO: 1.

In another aspect, the variant comprises or consists of a substitution at a position corresponding to position 398. In another aspect, the amino acid at a position corresponding to position 398 is substituted with Ala, Arg, Asn, Asp, Cys, Gln, Glu, Gly, His, Ile, Leu, Lys, Met, Phe, Pro, Ser, Thr, Trp, or Val. In another aspect, the variant comprises or consists of the substitution Y398E of the polypeptide of SEQ ID NO: 1.

In another aspect, the variant comprises or consists of a substitution at a position corresponding to position 401. In another aspect, the amino acid at a position corresponding to position 401 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q401H of the polypeptide of SEQ ID NO: 1.

In another aspect, the variant comprises or consists of a substitution at a position corresponding to position 402. In another aspect, the amino acid at a position corresponding to position 402 is substituted with Ala, Arg, Asn, Asp, Cys, Gln, Glu, Gly, Ile, Leu, Lys, Met, Phe, Pro, Ser, Thr, Trp, Tyr, or Val. In another aspect, the variant comprises or consists of the substitution H402S of the polypeptide of SEQ ID NO: 1.

In another aspect, the variant comprises or consists of a substitution at a position corresponding to position 403. In another aspect, the amino acid at a position corresponding to position 403 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D403A or D403E or D403T of the polypeptide of SEQ ID NO: 1.

In another aspect, the variant comprises or consists of a substitution at a position corresponding to position 405. In another aspect, the amino acid at a position corresponding to position 405 is substituted with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of the substitution F405C or F405N or F405S or F405T of the polypeptide of SEQ ID NO: 1.

In another aspect, the variant comprises or consists of a substitution at a position corresponding to position 406. In another aspect, the amino acid at a position corresponding to position 406 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D406V of the polypeptide of SEQ ID NO: 1.

In another aspect, the variant comprises or consists of a substitution at a position corresponding to position 407. In another aspect, the amino acid at a position corresponding to position 407 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H4070 or H407Q or H407T of the polypeptide of SEQ ID NO: 1.

In another aspect, the variant comprises or consists of a substitution at a position corresponding to position 407. In another aspect, the amino acid at a position corresponding to position 407 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H4070 or H407Q or H407T of the polypeptide of SEQ ID NO: 1.

In another aspect, the variant comprises or consists of a substitution at a position corresponding to position 41C. In another aspect, the amino acid at a position corresponding to position 410 is substituted with Ala, Arg, Asn, Asp, Cys, Gin, Glu, Gly, His, Leu, Lys, Met, Phe, Pro, Ser, Thr, Trp, Tyr, or Val. In another aspect, the variant comprises or consists of the substitution I41C H or I41C M or I41C R or I41C W of the polypeptide of SEQ ID NO: 1.

In another aspect, the variant comprises or consists of a substitution at a position corresponding to position 415. In another aspect, the amino acid at a position corresponding to position 415 is substituted with Ala, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution R4150 or R415L or R415M or R415Y of the polypeptide of SEQ ID NO: 1.

In another aspect, the variant comprises or consists of a substitution at a position corresponding to position 416. In another aspect, the amino acid at a position corresponding to position 416 is substituted with Ala, Arg, Asn, Asp, Cys, Gin, Gly, His, Ile, Leu, Lys, Met, Phe, Pro, Ser, Thr, Trp, Tyr, or Val. In another aspect, the variant comprises or consists of the substitution E416D or E416F or E416L or E416N of the polypeptide of SEQ ID NO: 1.

In another aspect, the variant comprises or consists of a substitution at a position corresponding to position 418. In another aspect, the amino acid at a position corresponding to position 418 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N418A or N418P or N418V of the polypeptide of SEQ ID NO: 1.

In another aspect, the variant comprises or consists of a substitution at a position corresponding to position 421. In another aspect, the amino acid at a position corresponding to position 421 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H421A or H421G or H421L or H421P or H421Q or H421Y of the polypeptide of SEQ ID NO: 1.

In another aspect, the variant comprises or consists of a substitution at a position corresponding to position 422. In another aspect, the amino acid at a position corresponding to position 422 is substituted with Ala, Arg, Asn, Asp, Cys, Gln, Glu, Gly, His, Ile, Leu, Lys, Met, Phe, Ser, Thr, Trp, Tyr, or Val. In another aspect, the variant comprises or consists of the substitution P422D or P422G or P422I or P422N or P422Q or P422S of the polypeptide of SEQ ID NO: 1.

In another aspect, the variant comprises or consists of a substitution at a position corresponding to position 423. In another aspect, the amino acid at a position corresponding to position 423 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N423G or N423H or N423I or N423L or N423M or N423P or N423Q or N423V or N423W or N423Y of the polypeptide of SEQ ID NO: 1.

In another aspect, the variant comprises or consists of a substitution at a position corresponding to position 433. In another aspect, the amino acid at a position corresponding to position 433 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G433A or G433N or G433S of the polypeptide of SEQ ID NO: 1.

In another aspect, the variant comprises or consists of a substitution at a position corresponding to position 434. In another aspect, the amino acid at a position corresponding to position 434 is substituted with Ala, Arg, Asn, Asp, Cys, Gin, Glu, Gly, His, Ile, Leu, Lys, Met, Phe, Ser, Thr, Trp, Tyr, or Val. In another aspect, the variant comprises or consists of the substitution P4341 or P434L of the polypeptide of SEQ ID NO: 1.

In another aspect, the variant comprises or consists of a substitution at a position corresponding to position 435. In another aspect, the amino acid at a position corresponding to position 435 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G435C or G435L or G435M or G435T or G435V of the polypeptide of SEQ ID NO: 1.

In another aspect, the variant comprises or consists of a substitution at a position corresponding to position 438. In another aspect, the amino acid at a position corresponding to position 438 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K438A or K438I or K438Q or K438S or K438V of the polypeptide of SEQ ID NO: 1.

In another aspect, the variant comprises or consists of a substitution at a position corresponding to position 439. In another aspect, the amino acid at a position corresponding to position 439 is substituted with Ala, Arg, Asn, Asp, Cys, Gin, Glu, Gly, His, Ile, Leu, Lys, Met, Phe, Pro, Ser, Thr, Tyr, or Val. In another aspect, the variant comprises or consists of the substitution W439A or W439D or W439I or W439K or W439L or W439N or W439S or W439T or W439V or W439Y of the polypeptide of SEQ ID NO: 1.

In another aspect, the variant comprises or consists of a substitution at a position corresponding to position 446. In another aspect, the amino acid at a position corresponding to position 446 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K446A or K446F of the polypeptide of SEQ ID NO: 1.

In another aspect, the variant comprises or consists of a substitution at a position corresponding to position 447. In another aspect, the amino acid at a position corresponding to position 447 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A447D or A447Y of the polypeptide of SEQ ID NO: 1.

In another aspect, the variant comprises or consists of a substitution at a position corresponding to position 449. In another aspect, the amino acid at a position corresponding to position 449 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q449T of the polypeptide of SEQ ID NO: 1.

In another aspect, the variant comprises or consists of a substitution at a position corresponding to position 450. In another aspect, the amino acid at a position corresponding to position 450 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V450D or V450N of the polypeptide of SEQ ID NO: 1.

In another aspect, the variant comprises or consists of a substitution at a position corresponding to position 452. In another aspect, the amino acid at a position corresponding to position 452 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H452L of the polypeptide of SEQ ID NO: 1.

In another aspect, the variant comprises or consists of a substitution at a position corresponding to position 455. In another aspect, the amino acid at a position corresponding to position 455 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T455C or T455M or T455V of the polypeptide of SEQ ID NO: 1.

In another aspect, the variant comprises or consists of a substitution at a position corresponding to position 457. In another aspect, the amino acid at a position corresponding to position 457 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N457D or N457E or N457I or N457L or N457V of the polypeptide of SEQ ID NO: 1.

In another aspect, the variant comprises or consists of a substitution at a position corresponding to position 458. In another aspect, the amino acid at a position corresponding to position 458 is substituted with Ala, Arg, Asn, Asp, Cys, Gin, Glu, Gly, His, Ile, Leu, Met, Phe, Pro, Ser, Thr, Trp, Tyr, or Val. In another aspect, the variant comprises or consists of the substitution K458D or K458L of the polypeptide of SEQ ID NO: 1.

In another aspect, the variant comprises or consists of a substitution at a position corresponding to position 460. In another aspect, the amino acid at a position corresponding to position 460 is substituted with Ala, Arg, Asn, Asp, Cys, Gin, Glu, His, Ile, Leu, Lys, Met, Phe, Pro, Ser, Thr, Trp, Tyr, or Val. In another aspect, the variant comprises or consists of the substitution G460M or G460V of the polypeptide of SEQ ID NO: 1.

In another aspect, the variant comprises or consists of a substitution at a position corresponding to position 461. In another aspect, the amino acid at a position corresponding to position 461 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T461F or T461I or T461Q or T461V of the polypeptide of SEQ ID NO: 1.

In another aspect, the variant comprises or consists of a substitution at a position corresponding to position 462. In another aspect, the amino acid at a position corresponding to position 462 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V462C of the polypeptide of SEQ ID NO: 1.

In another aspect, the variant comprises or consists of a substitution at a position corresponding to position 465. In another aspect, the amino acid at a position corresponding to position 465 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N465A or N465C or N465E or N465G or N465H or N465L or N465P or N465T of the polypeptide of SEQ ID NO: 1.

In another aspect, the variant comprises or consists of a substitution at a position corresponding to position 466. In another aspect, the amino acid at a position corresponding to position 466 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A466F or A466I or A466Y of the polypeptide of SEQ ID NO: 1.

In another aspect, the variant comprises or consists of a substitution at a position corresponding to position 467. In another aspect, the amino acid at a position corresponding to position 467 is substituted with Ala, Arg, Asn, Cys, Gin, Glu, Gly, His, Ile, Leu, Lys, Met, Phe, Pro, Ser, Thr, Trp, Tyr, or Val. In another aspect, the variant comprises or consists of the substitution D4671 of the polypeptide of SEQ ID NO: 1.

In another aspect, the variant comprises or consists of a substitution at a position corresponding to position 467. In another aspect, the amino acid at a position corresponding to position 467 is substituted with Ala, Arg, Asn, Asp, Cys, Gin, Glu, Gly, His, Ile, Leu, Lys, Met, Phe, Pro, Ser, Thr, Tyr, or Val. In another aspect, the variant comprises or consists of the substitution W469A or W469E or W469H or W469L or W469N or W469Q or W469R or W469S or W469Y of the polypeptide of SEQ ID NO: 1.

In another aspect, the variant comprises or consists of a substitution at a position corresponding to position 471. In another aspect, the amino acid at a position corresponding to position 471 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N471A or N471D or N471M or N471W of the polypeptide of SEQ ID NO: 1.

In another aspect, the variant comprises or consists of a substitution at a position corresponding to position 473. In another aspect, the amino acid at a position corresponding to position 473 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S473N of the polypeptide of SEQ ID NO: 1.

In another aspect, the variant comprises or consists of a substitution at a position corresponding to position 475. In another aspect, the amino acid at a position corresponding to position 475 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N475A or N475C or N475D or N475E or N475F or N475G or N475H or N475I or N475L or N475M or N475P or N475Q or N475S or N475T or N475V or N475W of the polypeptide of SEQ ID NO: 1.

In another aspect, the variant comprises or consists of a substitution at a position corresponding to position 476. In another aspect, the amino acid at a position corresponding to position 476 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G476F or G476L of the polypeptide of SEQ ID NO: 1.

In another aspect, the variant comprises or consists of a substitution at a position corresponding to position 477. In another aspect, the amino acid at a position corresponding to position 477 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G477A or G477H or G477Q or G477S or G477W of the polypeptide of SEQ ID NO: 1.

In another aspect, the variant comprises or consists of a substitution at a position corresponding to position 478. In another aspect, the amino acid at a position corresponding to position 478 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution S478A or S478F or S478Q of the polypeptide of SEQ ID NO: 1.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 1 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 2 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17. In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 3 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 4 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 5 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 6 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 7 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 8 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 9 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 10 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 11 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 12 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 13 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 14 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 15 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 16 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and wherein said alpha-amylase variant has an increased specific activity measured as Improvement Factor (IF) of ≥1.1 compared to parent polypeptide of SEQ ID NO: 17 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q; using SEQ ID NO: 1 for numbering, and wherein said alpha-amylase variant has an increased specific activity measured as Improvement Factor (IF) of ≥1.1 compared to parented polypeptide of SEQ ID NO: 1 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variants of the present invention comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q; using SEQ ID NO: 1 for numbering, and wherein said alpha-amylase variant has an increased specific activity measured as Improvement Factor (IF) of ≥1.1 compared to parented polypeptide of SEQ ID NO: 2 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q; using SEQ ID NO: 1 for numbering, and wherein said alpha-amylase variant has an increased specific activity measured as Improvement Factor (IF) of ≥1.1 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitutions at position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, TBS, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q; using SEQ ID NO: 1 for numbering, and wherein said alpha-amylase variant has an increased specific activity in Model A detergent composition measured as Improvement Factor (IF) of ≥1.1 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitutions at position corresponding to positions N3A; N3C; N3D; N3E; N3F; N3G; N3H; N3L; N3P; N3Q; N3S; N3T; N3V; T5E; G7E; G7F; G7H; G7K; G7L; G7P; G7R; G7S; G7T; G7V; G7W; T8S; H16D; H16R; P18D; P18N; N25T; R26Y; D29N; D30F; R37A; R37N; T40A; T400; T40D; T40E; T40G; T40H; T40I; T40K; T40N; T40V; T40W; A41C; A41D; A41E; A41H; A41I; A41K; A41N; A41R; A41T; A41V; A41Y; W43E; P46A; G50M; G50P; G50Q; G57A; G57S; A60E; A60S; A60V; E68F; Q71F; G73D; G73H; G731; G73L; G73Q; G73S; G73T; G73W; K78S; K78T; T81M; Q841; Q84W; Q84Y; E86K; E86S; S87G; 587L; S87P; H90P; A91Q; A91V; A91W; K93V; V97C; V97H; Q98E; Q98F; Q98G; Q981; Q98K; Q98L; Q98M; Q98R; Q98T; Q98Y; V103C; V103G; V103S; M105F; G110K; G110R; A111E; A111Q; A113T; T114M; T114V; N116Y; V117F; A119H; A119L; A119Y; V120E; E121I; N125S; N126I; N128E; Q129T; I131Y; S132A; S132D; S132H; S132I; S132K; S132L; S132P; S132R; S132T; S132V; S132Y; G133E; G133L; G133T; G133V; Y135C; Y135D; Y135E; Y135P; I137D; I137N; I137Q; I137W; I137Y; E138S; E138V; A139L; W140A; W140D; W140M; W140N; W140P; W140S; W140T; W140V; T141G; K142E; K142N; F143H; F143I; F143M; F145G; F145H; F145I; F145L; F145S; P146A; P146H; P146N; G147D; G149F; G149K; W159A; H161W; F162T; V165P; W167D; W167G; W167P; Q169V; Q169Y; R171P; R176E; R176Q; R176V; R176Y; I177H; I177P; K179A; K179M; K179V; F180D; F180G; R181D; R181E; R181G; R181M; R181Y; K185V; K185W; A186D; W187T; N195Y; G196D; N197V; N197Y; A204F; A204H; A204L; M208D; P211C; P211M; E216L; R219V; T225D; T225F; T225H; T225K; T225L; T225N; T225R; T225Y; L228V; L230C; L230F; D231G; D231T; D231V; I235A; K242L; Y243D; Y243M; Y243N; R247G; R247P; L250S; L250Y; T251D; H252P; V253F; V253S; R254D; R254F; R254H; R254P; R254T; R254V; R254W; N255C; N255F; N255P; T257A; T257C; T257D; T257E; T257G; T257P; T257Q; T257S; T257W; T257Y; K259P; K259V; K259W; K259Y; E260V; M261D; M261F; M261P; M261R; M261S; M261W; F262A; F262C; F262E; F262H; F262I; F262K; F262L; F262N; F262P; F262Q; F262R; F262S; F262T; F262V; F262W; A263E; A263H; A263W; A265Q; L272W; G273Q; L279I; N280P; K281H; K281I; K281S; N283P; W284A; W284D; W284E; W284G; W2841; W284K; W284M; W284N; W284P; W284Q; W284S; W284T; W284V; W284Y; S2871; S287K; S287L; S287P; S287R; S287T; S287V; S287Y; P292L; P292M; P292Y; H294N; H294S; N296T; Y298D; Y298E; N299T; A300G; S303A; S303C; S303D; S303E; S303F; S303G; S303H; S303L; S303M; S303N; S303Q; S303T; S303V; S303Y; G305E; Y307S; A310D; A310N; K311A; K311C; L313Q; N314P; N314S; G315E; G315M; G315V; Q319C; Q319E; K320S; K320W; H321F; H321K; H321L; H321R; H321T; H321V; H321W; P322C; P322E; P322H; P322T; P322Y; M323A; M323C; M323D; M323E; M323G; M323H; M323L; M323N; M323P; M323R; M323S; M323Y; H324A; H324I; H324P; H324T; H324W; H324Y; S334V; G337V; L340M; S342A; S342G; S342T; V344A; V344C; V344I; Q345N; E346A; W347A; W347E; W347F; W347G; W347H; W347K; W347N; W347P; W347R; P350A; P350C; P350N; P350T; R359C; R359F; R359G; R359M; R359W; E360D; E360H; E360P; E360T; E360W; Q361D; Q361H; Q3611; Q361K; Q361L; Q361M; Q361N; Q361P; Q361R; Q361S; Q361T; Q361V; Q361W; Q361Y; G362M; Y363A; Y363E; Y363F; Y363G; Y3631; Y363L; Y363Q; Y363S; Y363T; Y363V; Y363W; P364C; Y368C; Y368Q; Y368R; Y368T; Y372C; Y372G; Y372N; Y372T; P375Q; P375T; P380F; M3821; K383C; K383L; K383P; K383T; A384D; A384N; K385N; K385V; D387C; D387P; D387R; D387W; P3881; E391M; R393K; R393M; N395C; N395D; N395K; N395V; N395W; F396E; F396G; F396T; A397V; Y398E; Q401H; H402S; D403A; D403E; D403T; F405C; F405N; F405S; F405T; D406V; H4070; H407Q; H407T; I41C H; I41C M; I41C R; I41C W; R415C; R415L; R415M; R415Y; E416D; E416F; E416L; E416N; N418A; N418P; N418V; H421A; H421G; H421L; H421P; H421Q; H421Y; P422D; P422G; P4221; P422N; P422Q; P422S; N423G; N423H; N423I; N423L; N423M; N423Q; N423V; N423W; N423Y; G433A; G433S; P434I; P434L; G435C; G435L; G435M; G435T; G435V; K438A; K4381; K438Q; K438S; K438V; W439A; W439D; W439I; W439K; W439L; W439N; W439S; W439T; W439V; W439Y; K446A; K446F; A447D; A447Y; Q449T; V450D; V450N; H452L; T455C; T455M; T455V; N457D; N457E; N457I; N457L; N457V; K458D; K458L; G460M; T461F; T461I; T461Q; T461V; V462C; N465A; N465C; N465E; N465G; N465H; N465L; N465P; N465T; A466F; A466I; A466Y; D4671; W469A; W469E; W469H; W469L; W469N; W469Q; W469R; W469Y; N471A; N471D; N471M; N471W; S473N; N475A; N475C; N475D; N475E; N475F; N475G; N475H; N475I; N475L; N475M; N475P; N475Q; N475S; N475T; N475V; N475W; G476F; G476L; G477A; G477H; G477Q; G477S; G477W; S478A; S478F; and S478Q, using SEQ ID NO: 1 for numbering, and wherein said alpha-amylase variant has an increased specific activity in Model A detergent composition measured as Improvement Factor (IF) of ≥1.2 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitutions at position corresponding to positions N3A; N3C; N3D; N3E; N3F; N3G; N3H; N3L; N3P; N3Q; N3V; T5E; G7E; G7F; G7H; G7L; G7R; G7S; G7T; G7V; G7W; H16D; R26Y; D29N; D30F; R37A; R37N; T40A; T400; T40D; T40E; T40G; T40H; T40I; T40N; T40V; T40W; A41C; A41D; A41E; A41H; A41I; A41K; A41N; A41R; A41Y; P46A; G50Q; G57A; G57S; A60S; A60V; E68F; G73D; G73H; G731; G73L; G73Q; G73S; G73T; G73W; K78T; T81M; Q841; Q84W; E86S; S87G; H90P; Q98F; Q981; Q98K; Q98L; Q98M; Q98Y; V103C; V103G; G110K; G110R; A111E; A111Q; A119L; A119Y; V120E; N125S; N128E; I131Y; S132A; S132D; S132H; S132I; S132K; S132L; S132R; S132V; S132Y; G133E; Y135C; Y135D; Y135E; Y135P; I137N; I137W; E138S; E138V; A139L; W140A; W140D; W140M; W140N; W140P; W140S; W140T; K142E; F143H; F143I; F145I; P146A; P146H; P146N; G147D; G149K; H161W; V165P; W167D; W167P; Q169V; R171P; R176E; R176Q; R176Y; I177H; K179A; K179V; R181E; R181G; R181M; R181Y; A186D; W187T; G196D; N197V; N197Y; A204H; A204L; P211C; P211M; T225D; T225F; T225H; T225K; T225L; T225N; T225R; T225Y; L230C; L230F; D231V; Y243D; Y243N; R247G; R247P; L250S; H252P; V253F; R254T; R254V; R254W; N255P; T257A; T257C; T257D; T257E; T257G; T257W; K259P; K259V; K259W; E260V; M261D; M261F; M261P; M261R; M261S; M261W; F262A; F262C; F262E; F262H; F262I; F262K; F262L; F262N; F262P; F262Q; F262R; F262S; F262T; F262V; F262W; A263E; A263H; A263W; A265Q; L279I; K281I; N283P; W284A; W284D; W284E; W284G; W2841; W284K; W284M; W284N; W284P; W284Q; W284S; W284T; W284V; W284Y; S287K; S287L; S287R; S287T; S287V; S287Y; H294S; N296T; Y298D; N299T; S303A; S303C; S303D; S303E; S303F; S303G; S303H; S303L; S303M; S303N; S303Q; S303T; S303V; S303Y; G305E; Y307S; A310N; K311A; K311C; L313Q; N314P; N314S; G315E; G315V; Q319E; K320W; H321F; H321K; H321L; H321V; P322C; P322E; P322H; P322T; P322Y; M323A; M323C; M323D; M323E; M323G; M323H; M323L; M323N; M323P; M323R; M323S; M323Y; H324A; H324I; H324P; H324T; H324W; H324Y; G337V; L340M; S342G; V344C; E346A; W347E; W347F; W347G; W347H; W347K; W347N; W347P; W347R; P350C; P350N; P350T; R359G; R359W; E360D; E360T; E360W; Q361D; Q361H; Q3611; Q361K; Q361L; Q361M; Q361N; Q361P; Q361R; Q361T; Q361V; Q361W; Q361Y; G362M; Y363A; Y363F; Y363G; Y3631; Y363Q; Y363S; Y363T; Y363V; Y363W; P364C; Y368C; Y368Q; Y368R; Y372C; Y372G; P375Q; P375T; P380F; K383C; K383T; A384D; K385N; K385V; D387C; D387P; D387R; E391M; R393K; R393M; N395C; N395D; N395W; F396E; F396G; F396T; Y398E; H402S; D403A; D403T; F405C; F405N; F405S; F405T; D406V; H4070; H407Q; H407T; I41C H; I41C M; I41C W; R415C; R415L; R415M; R415Y; E416F; E416L; E416N; N418A; N418P; H421A; H421G; H421L; H421P; H421Q; H421Y; P422D; P422G; P422N; P422S; N423G; N423I; N423L; N423M; N423Q; N423V; N423W; N423Y; G433S; P434I; P434L; G435M; G435T; G435V; K4381; K438Q; K438S; K438V; W439A; W439D; W439I; W439K; W439L; W439N; W439S; W439T; W439V; K446A; K446F; A447D; A447Y; H452L; T455V; N457D; N457E; N457I; N457L; N457V; K458D; K458L; G460M; T461F; T461I; T461Q; T461V; V462C; N465A; N465E; N465G; N465L; A466F; A466I; A466Y; D4671; W469A; W469E; W469H; W469L; W469Q; W469R; W469Y; N471A; N471D; N471M; N475A; N475C; N475D; N475E; N475F; N475G; N475H; N475I; N475L; N475M; N475P; N475Q; N475S; N475T; N475V; N475W; G476L; G477A; G477H; G477Q; G477S; G477W; S478A; and S478F; using SEQ ID NO: 1 for numbering, and wherein said alpha-amylase variant has an increased specific activity in Model A detergent composition measured as Improvement Factor (IF) of ≥1.3 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitutions at position corresponding to positions N3A; N3C; N3D; N3E; N3F; N3G; N3H; N3L; N3V; G7F; G7H; G7R; G7V; G7W; R26Y; D29N; R37A; R37N; T400; T40D; T40E; T40G; T40I; T40N; T40V; A41C; A41D; A41E; A41K; A41N; A41R; A41Y; G73D; G73H; G731; G73L; G73Q; G73S; G73T; G73W; K78T; Q841; Q84W; S87G; Q981; Q98L; V103C; G110K; A111Q; A119Y; N125S; N128E; S132D; S132I; S132L; S132R; Y135C; Y135E; I137W; E138S; E138V; A139L; W140A; W140D; W140M; W140N; W140P; W140S; W140T; K142E; F143I; F145I; P146H; G149K; H161W; V165P; W167D; W167P; Q169V; R176Q; I177H; P211C; T225F; T225H; T225K; T225R; T225Y; L230C; Y243D; R247G; V253F; N255P; T257A; T257D; T257E; K259P; K259V; M261D; M261R; M261W; F262A; F262C; F262E; F262H; F262I; F262K; F262L; F262N; F262P; F262Q; F262R; F262S; F262W; A263H; L279I; N283P; W284D; W284E; W284I; W284K; W284M; W284N; W284P; W284Q; W284S; W284V; S287K; S287L; S287R; S287Y; N296T; S303A; S303C; S303D; S303E; S303F; S303G; S303H; S303M; S303N; S303Q; S303T; S303V; S303Y; G305E; K311C; L313Q; N314P; G315V; H321K; H321L; H321V; P322C; P322E; P322H; P322T; P322Y; M323A; M323D; M323E; M323G; M323L; M323P; M323S; M323Y; H324A; H324I; H324P; H324W; L340M; S342G; E346A; W347F; W347K; W347N; W347P; W347R; R359W; E360W; Q361D; Q361H; Q3611; Q361K; Q361L; Q361M; Q361P; Q361R; Q361V; Q361W; Q361Y; G362M; Y363A; Y363G; Y363T; Y363V; Y363W; P364C; Y368C; Y368Q; Y372C; Y372G; K383T; A384D; D387C; D387P; E391M; R393K; R393M; N395D; N395W; F396E; F396T; D403A; F405C; F405N; F405S; D406V; H4070; H407T; I41C H; I41C M; I41C W; R415C; R415L; R415M; R415Y; N418P; H421A; H421L; H421P; H421Q; P422D; P422G; N423L; N423V; N423W; N423Y; G433S; G435M; K438V; W439A; W439I; W439K; W439S; W439T; W439V; K446A; A447Y; H452L; T455V; N457E; N457I; N457V; K458D; T461F; T461Q; N465A; N465E; N465G; A466I; D4671; W469A; W469E; W469Y; N471A; N471D; N471M; N475A; N475C; N475D; N475E; N475F; N475G; N475H; N475L; N475M; N475P; N475Q; N475S; N475T; N475V; N475W; G477A; G477H; G477Q; G477S; G477W; S478A; using SEQ ID NO: 1 for numbering, and wherein said alpha-amylase variant has an increased specific activity in Model A detergent composition measured as Improvement Factor (IF) of ≥1.4 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitutions at position corresponding to positions N3A; N3C; N3D; N3E; N3G; N3L; G7F; R26Y; R37A; R37N; T400; T40D; T40G; T40I; T40N; T40V; A41C; A41D; A41K; A41N; A41R; G73D; G731; G73L; G73Q; G73S; G73T; G73W; K78T; Q841; G110K; A111Q; N128E; Y135E; A139L; W140A; W140M; K142E; F145I; G149K; V165P; W167P; R176Q; I177H; T225F; T225H; T225K; T225Y; L230C; R247G; T257D; T257E; M261W; F262A; F262E; F262K; F262N; F262P; F262Q; F262R; F262S; F262W; N283P; W284D; W284P; W284S; W284V; S287Y; S303A; S303C; S303D; S303E; S303F; S303G; S303H; S303M; S303N; S303Q; S303T; S303V; S303Y; L313Q; N314P; H321K; H321L; H321V; P322C; P322H; P322Y; M323A; M323D; M323Y; H324P; H324W; L340M; S342G; E346A; W347N; W347P; W347R; R359W; E360W; Q361K; Q361L; Q361M; Q361R; Q361W; Q361Y; Y363G; Y363V; Y363W; Y368C; Y372C; K383T; D387P; E391M; R393K; R393M; N395D; N395W; D403A; F405C; F405S; D406V; H407C; H407T; I41C H; R415C; R415L; R415M; R415Y; N418P; H421A; H421L; H421P; P422D; N423L; N423V; G435M; W439A; K446A; H452L; T455V; N457I; K458D; N465E; N471A; N471D; N471M; N475A; N475C; N475D; N475E; N475F; N475G; N475H; N475L; N475P; N475Q; N475V; N475W; G477A; G477H; G477Q; and G477S; using SEQ ID NO: 1 for numbering, and wherein said alpha-amylase variant has an increased specific activity in Model A detergent composition measured as Improvement Factor (IF) of ≥1.5 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprises one or more of the following substitutions at position corresponding to positions R37A; R176Q; T225Y; F262P; N283P; S303C; S303D; S303G; S303M; S303N; S303T; S303V; S303Y Y368C; N395D; H407T; I41C H; R415C; H421A; G435M; N475C; G477A; and G477H; using SEQ ID NO: 1 for numbering, and wherein said alpha-amylase variant has an increased specific activity in Model A detergent composition measured as Improvement Factor (IF) of ≥2.0 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention comprise one or more of the following substitutions at position corresponding to positions S303C; S303T; Y368C; and H421A; using SEQ ID NO: 1 for numbering, and wherein said alpha-amylase variant has an increased specific activity in Model A detergent composition measured as Improvement Factor (IF) of ≥2.5 and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In another aspect, the alpha-amylase variant of the present invention further comprise a deletion at two or more positions corresponding to positions R181, G182, D183, and G184, using SEQ ID NO: 1 for numbering and further wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17.

In one aspect, the alpha-amylase variant of the present invention wherein the deletion is selected from the group consisting of R181*+G182*, R181*+D183*, R181*+G184*, G182*+D183*, G182*+G184*, or D183*+G184*, preferably D183*+G184*.

The variant may further comprise one or more additional substitution and/or deletion at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Therefore even though the alpha-amylase variant according to the invention may only comprise one specific substitution providing the improved property according to the invention it may still have addition modifications leading to a alpha-amylase variant having at least 80%, at least 85%, at least 90%, at least 91%, at least 92%, at least 93%, at least 94%, at least 95%, at least 96%, at least 97%, at least 98%, or at least 99%, but less than 100% sequence identity, to the amino acid sequence of the SEQ ID Nos: 1, 2, 3, 4, 5, 6, 7, 8, 9, 10, 11, 12, 13, 14, 15, 16, or 17. These additional modification should preferably not significantly change the improved properties of the alpha-amylase variant.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64.

In an embodiment, the variant has improved (increased) specific activity compared to the parent polypeptide.

In an embodiment, the variant has improved (increased) specific activity compared to the parent polypeptide, preferably of SEQ ID NO: 1 and/or SEQ ID NO:2.

In an embodiment, the variant has improved (increased) stability under storage conditions compared to the parent polypeptide, preferably of SEQ ID NO: 1 and/or SEQ ID NO:2.

In an embodiment, the variant has improved (increased) thermostability compared to the parent polypeptide, preferably of SEQ ID NO: 1 and/or SEQ ID NO:2.

In an embodiment, the variant has improved (increased) wash performance compared to the parent polypeptide, preferably of SEQ ID NO: 1 and/or SEQ ID NO:2.

### Parent Alpha-Amylases

The parent alpha-amylase may be a polypeptide with at least 60% sequence identity with any one of the polypeptides of SEQ ID NOs 1, 2, 3, 4, 5, 6, 7, 8, 9, 10, 11, 12, 13, 14, 15, 16 or 17.

In one embodiment, the parent has a sequence identity to the polypeptide of SEQ ID NO: 1 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 1.

The parent preferably comprises or consists of the amino acid sequence of SEQ ID NO: 1. In one embodiment the parent comprises or consists of the polypeptide of SEQ ID NO: 1. In another embodiment, the parent is an allelic variant of the polypeptide of SEQ ID NO: 1.

In one embodiment, the parent has a sequence identity to the polypeptide of SEQ ID NO: 2 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 2.

The parent preferably comprises or consists of the amino acid sequence of SEQ ID NO: 2. In one embodiment the parent comprises or consists of the polypeptide of SEQ ID NO: 2. In another embodiment, the parent is an allelic variant of the polypeptide of SEQ ID NO: 2.

In one embodiment, the parent has a sequence identity to the polypeptide of SEQ ID NO: 3 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 3.

The parent preferably comprises or consists of the amino acid sequence of SEQ ID NO: 3. In one embodiment the parent comprises or consists of the polypeptide of SEQ ID NO: 3. In another embodiment, the parent is an allelic variant of the polypeptide of SEQ ID NO: 3. In one embodiment, the parent has a sequence identity to the polypeptide of SEQ ID NO: 4 of at least 60% e.g., at least 65%, at least 70%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 4.

The parent preferably comprises or consists of the amino acid sequence of SEQ ID NO: 4. In one embodiment the parent comprises or consists of the polypeptide of SEQ ID NO: 4. In another embodiment, the parent is an allelic variant of the polypeptide of SEQ ID NO: 4. In one embodiment, the parent has a sequence identity to the polypeptide of SEQ ID NO: 5 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 5.

The parent preferably comprises or consists of the amino acid sequence of SEQ ID NO: 5. In one embodiment the parent comprises or consists of the polypeptide of SEQ ID NO: 5. In another embodiment, the parent is an allelic variant of the polypeptide of SEQ ID NO: 5.

In one embodiment, the parent has a sequence identity to the polypeptide of SEQ ID NO: 6 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 6.

The parent preferably comprises or consists of the amino acid sequence of SEQ ID NO: 6. In one embodiment the parent comprises or consists of the polypeptide of SEQ ID NO: 6. In another embodiment, the parent is an allelic variant of the polypeptide of SEQ ID NO: 6.

In one embodiment, the parent has a sequence identity to the polypeptide of SEQ ID NO: 7 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 7.

The parent preferably comprises or consists of the amino acid sequence of SEQ ID NO: 7. In one embodiment the parent comprises or consists of the polypeptide of SEQ ID NO: 7. In another embodiment, the parent is an allelic variant of the polypeptide of SEQ ID NO: 7.

In one embodiment, the parent has a sequence identity to the polypeptide of SEQ ID NO: 8 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 8.

The parent preferably comprises or consists of the amino acid sequence of SEQ ID NO: 8. In one embodiment the parent comprises or consists of the polypeptide of SEQ ID NO: 8. In another embodiment, the parent is an allelic variant of the polypeptide of SEQ ID NO: 8.

In one embodiment, the parent has a sequence identity to the polypeptide of SEQ ID NO: 9 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 9.

The parent preferably comprises or consists of the amino acid sequence of SEQ ID NO: 9. In one embodiment the parent comprises or consists of the polypeptide of SEQ ID NO: 9. In another embodiment, the parent is an allelic variant of the polypeptide of SEQ ID NO: 9.

In one embodiment, the parent has a sequence identity to the polypeptide of SEQ ID NO: 10 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 10.

The parent preferably comprises or consists of the amino acid sequence of SEQ ID NO: 10. In one embodiment the parent comprises or consists of the polypeptide of SEQ ID NO: 10. In another embodiment, the parent is an allelic variant of the polypeptide of SEQ ID NO:10.

In one embodiment, the parent has a sequence identity to the polypeptide of SEQ ID NO: 11 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 11.

The parent preferably comprises or consists of the amino acid sequence of SEQ ID NO: 11. In one embodiment the parent comprises or consists of the polypeptide of SEQ ID NO: 11. In another embodiment, the parent is an allelic variant of the polypeptide of SEQ ID NO: 11.

In one embodiment, the parent has a sequence identity to the polypeptide of SEQ ID NO: 12 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 12.

The parent preferably comprises or consists of the amino acid sequence of SEQ ID NO: 12. In one embodiment the parent comprises or consists of the polypeptide of SEQ ID NO: 12. In another embodiment, the parent is an allelic variant of the polypeptide of SEQ ID NO: 12.

In one embodiment, the parent has a sequence identity to the polypeptide of SEQ ID NO: 13 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 13.

The parent preferably comprises or consists of the amino acid sequence of SEQ ID NO: 13. In one embodiment the parent comprises or consists of the polypeptide of SEQ ID NO: 13. In another embodiment, the parent is an allelic variant of the polypeptide of SEQ ID NO: 13.

In one embodiment, the parent has a sequence identity to the polypeptide of SEQ ID NO: 14 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 14.

The parent preferably comprises or consists of the amino acid sequence of SEQ ID NO: 14. In one embodiment the parent comprises or consists of the polypeptide of SEQ ID NO: 14. In another embodiment, the parent is an allelic variant of the polypeptide of SEQ ID NO: 14.

In one embodiment, the parent has a sequence identity to the polypeptide of SEQ ID NO: 15 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 15.

The parent preferably comprises or consists of the amino acid sequence of SEQ ID NO: 15. In one embodiment the parent comprises or consists of the polypeptide of SEQ ID NO: 15. In another embodiment, the parent is an allelic variant of the polypeptide of SEQ ID NO: 15.

In one embodiment, the parent has a sequence identity to the polypeptide of SEQ ID NO: 16 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 16.

The parent preferably comprises or consists of the amino acid sequence of SEQ ID NO: 16. In one embodiment the parent comprises or consists of the polypeptide of SEQ ID NO: 16. In another embodiment, the parent is an allelic variant of the polypeptide of SEQ ID NO: 16.

In one embodiment, the parent has a sequence identity to the polypeptide of SEQ ID NO: 17 of at least 60% e.g., at least 65%, at least 70%, at least 75%, at least 80%, at least 85%, at least 87%, at least 90%, at least 95%, at least 96%, at least 97%, at least 98%, at least 99%, or 100%, which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 17.

The parent preferably comprises or consists of the amino acid sequence of SEQ ID NO: 17. In one embodiment the parent comprises or consists of the polypeptide of SEQ ID NO: 17. In another embodiment, the parent is an allelic variant of the polypeptide of SEQ ID NO: 17.

The amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or an active fragment thereof, may be used to design nucleic acid probes to identify and clone DNA encoding a parent from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, e.g., at least 25, at least 35, or at least 70 nucleotides in length. Preferably, the nucleic acid probe is at least 100 nucleotides in length or at least 200 nucleotides in length, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may be screened for DNA that hybridizes with the probes described above and encodes a parent. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material, which is used in a Southern blot.

For purposes of the present invention, hybridization indicates that the polynucleotide hybridizes to a labeled nucleotide probe corresponding to a polynucleotide encoding SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or a subsequence thereof, under low to very high stringency conditions. Molecules to which the probe hybridizes can be detected using, for example, X-ray film or any other detection means known in the art.

In one aspect, the nucleic acid probe is a polynucleotide that encodes the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or an active fragment thereof.

For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), 50° C. (low stringency), 55° C. (medium stringency), 60° C. (medium-high stringency), 65° C. (high stringency), or 70° C. (very high stringency).

For short probes that are about 15 nucleotides to about 70 nucleotides in length, stringency conditions are defined as prehybridization and hybridization at about 5° C. to about 10° C. below the calculated Tₘ using the calculation according to Bolton and McCarthy (1962, Proc. Natl. Acad. Sci. USA 48: 1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally. The carrier material is finally washed once in 6X SCC plus 0.1% SDS for 15 minutes and twice each for 15 minutes using 6×SSC at 5° C. to 10° C. below the calculated Tₘ. The parent may be obtained from microorganisms of any genus. For purposes of the present invention, the term “obtained from” as used herein in connection with a given source shall mean that the parent encoded by a polynucleotide is produced by the source or by a cell in which the polynucleotide from the source has been inserted. In one aspect, the parent is secreted extracellularly.

The parent may be a bacterial alpha-amylase. For example, the parent may be a gram-positive bacterial polypeptide such as a Bacillus, Clostridium, Enterococcus, Geobacillus, Lactobacillus, Lactococcus, Oceanobacillus, Staphylococcus, Streptococcus, or Streptomyces alpha-amylase, or a gram-negative bacterial polypeptide such as a Campylobacter, E. coli, Flavobacterium, Fusobacterium, Helicobacter, Ilyobacter, Neisseria, Pseudomonas, Salmonella, or Ureaplasma alpha-amylase.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis alpha-amylase.

In another aspect, the parent is a Streptococcus equisimilis, Streptococcus pyogenes, Streptococcus uberis, or Streptococcus equi subsp. Zooepidemicus alpha-amylase.

In another aspect, the parent is a Streptomyces achromogenes, Streptomyces avermitilis, Streptomyces coelicolor, Streptomyces griseus, or Streptomyces lividans alpha-amylase. The parent may be a fungal alpha-amylase. For example, the parent may be a yeast alpha-amylase such as a Candida, Kluyveromyces, Pichia, Saccharomyces, Schizosaccharomyces, or Yarrowia alpha-amylase. For example, the parent may be a filamentous fungal alpha-amylase such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria alpha-amylase.

In another aspect, the parent is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis alpha-amylase.

In another aspect, the parent is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride alpha-amylase.

In another aspect, the parent is a Bacillus sp. alpha-amylase, e.g., the alpha-amylase of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc,) using the above-mentioned probes. Techniques for isolating microorganisms and DNA directly from natural habitats are well known in the art. The polynucleotide encoding a parent may then be derived by similarly screening a genomic or cDNA library of another microorganism or mixed DNA sample. Once a polynucleotide encoding a parent has been detected with a probe(s), the polynucleotide may be isolated or cloned by utilizing techniques that are known to those of ordinary skill in the art (see, e.g., Sambrook et al., 1989, supra).

The parent may be a hybrid polypeptide in which a portion of one polypeptide is fused at the N-terminus or the C-terminus of a portion of another polypeptide.

The parent may also be a fused polypeptide or cleavable fusion polypeptide in which one polypeptide is fused at the N-terminus or the C-terminus of another polypeptide. A fused polypeptide is produced by fusing a polynucleotide encoding one polypeptide to a polynucleotide encoding another polypeptide. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Preparation of Variants

The present invention relates to methods for obtaining a variant having alpha-amylase activity, comprising (a) introducing into a parent alpha-amylase a substitution at one or more positions corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and said variant has alpha-amylase activity; and (b) recovering said variant.

In one aspect, the invention relates to a method for obtaining a variant having alpha-amylase activity, comprising (a) introducing into a parent alpha-amylase a substitution at one or more positions corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, and said variant has alpha-amylase activity; and (b) recovering said variant.

In another embodiment, the invention relates to a method for obtaining a variant having alpha-amylase activity, comprising (a) introducing into a parent alpha-amylase a substitution at one or more positions, wherein the substitution is selected from N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering and (b) recovering the variant.

The variants may be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (several) mutations are created at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests at the plasmid and the oligonucleotide is the same, permitting sticky ends of the plasmid and insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to isolated polynucleotides that encode any of the variants of the present invention. Accordingly, the present invention relates to isolated polynucleotides encoding a variant comprising a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO:1 for numbering, and wherein the variant has at least 60%, at least 65%, at least 70%, at least 75%, at least 80%, such as at least 85%, at least 90%, such as at least 95%, such as at least 97%, but less than 100% sequence identity with the amino acid sequence set forth in SEQ ID NOs: 1, 2, 3, 4, 5, 6, 7, 8, 9, 10, 11, 12, 13, 14, 15, 16 or 17 and wherein the variant has alpha-amylase activity.

In one aspect, polynucleotide sequence of SEQ ID NO: 18 encodes polypeptide SEQ ID NO: 1 and/or SEQ ID NO: 2.

### Nucleic Acid Constructs

The present invention also relates to nucleic acid constructs comprising a polynucleotide encoding a variant of the present invention operably linked to one or more (several) control sequences that direct the expression of the coding sequence in a suitable host cell under conditions compatible with the control sequences. Accordingly, the present invention relates to nucleic acid constructs comprising a polynucleotide encoding a variant comprising a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, wherein the polynucleotide is operably linked to one or more control sequences that direct the expression of the coding sequence in a suitable host cell under conditions compatible with the control sequences.

A polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter sequence, which is recognized by a host cell for expression of the polynucleotide. The promoter sequence contains transcriptional control sequences that mediate the expression of the variant. The promoter may be any nucleic acid sequence that shows transcriptional activity in the host cell including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, E. coli lac operon, Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra.

Examples of suitable promoters for directing the transcription of the nucleic acid constructs of the present invention in a filamentous fungal host cell are the promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter including a gene encoding a neutral alpha-amylase in Aspergilli in which the untranslated leader has been replaced by an untranslated leader from a gene encoding triose phosphate isomerase in Aspergilli; non-limiting examples include modified promoters including the gene encoding neutral alpha-amylase in Aspergillus niger in which the untranslated leader has been replaced by an untranslated leader from the gene encoding triose phosphate isomerase in Aspergillus nidulans or Aspergillus oryzae); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the variant. Any terminator that is functional in the host cell may be used.

Preferred terminators for filamentous fungal host cells are obtained from the genes for Aspergillus nidulans anthranilate synthase, Aspergillus niger alpha-glucosidase, Aspergillus niger glucoamylase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA that is important for translation by the host cell. The leader sequence is operably linked to the 5′-terminus of the polynucleotide encoding the variant. Any leader sequence that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding region naturally linked in translation reading frame with the segment of the coding region that encodes the variant. Alternatively, the 5′-end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the variant. However, any signal peptide coding region that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding region that encodes a propeptide positioned at the N-terminus of a variant. The resultant polypeptide is known as a proenzyme or propolypeptide (or a zymogen in some cases). A propolypeptide is generally inactive and can be converted to an active polypeptide by cat alytic or autocat alytic cleavage of the propeptide from the propolypeptide. The propeptide coding region may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide regions are present at the N-terminus of a variant, the propeptide region is positioned next to the N-terminus of the variant and the signal peptide region is positioned next to the N-terminus of the propeptide region.

It may also be desirable to add regulatory sequences that allow the regulation of the expression of the variant relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

### Expression Vectors

The present invention also relates to recombinant expression vectors comprising a polynucleotide of the present invention, a promoter, and transcriptional and translational stop signals. Accordingly, the present invention relates to recombinant vectors comprising a polynucleotide encoding a variant comprising a substitution at one or more positions corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1, a promoter, and transcriptional and translational stop signals. The various nucleotide and control sequences may be joined together to produce a recombinant expression vector that may include one or more (several) convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about the expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably comprises one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are the dal genes from Bacillus licheniformis or Bacillus subtilis, or markers that confer antibiotic resistance such as ampicillin, chloramphenicol, kanamycin, or tetracycline resistance. Suitable markers for yeast host cells are ADE2, HIS3, LEU2, LYS2, MET3, TRP1, and URA3.

The vector preferably comprises an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may comprise additional nucleotide sequences for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of identity to the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a nucleotide sequence that enables a plasmid or vector to replicate in vivo.

More than one copy of a polynucleotide of the present invention may be inserted into the host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra) to obtain substantially pure variants.

### Host Cells

The present invention also relates to recombinant host cells, comprising a polynucleotide of the present invention operably linked to one or more (several) control sequences that direct the production of a variant of the present invention. Accordingly, the present invention relates to recombinant host cells, comprising a polynucleotide encoding a variant comprising a substitution at one or more positions corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, wherein the polynucleotide is operably linked to one or more control sequences that direct the production of a variant. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any gram-positive or gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell, including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell, including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may, for instance, be effected by protoplast transformation (see, e.g., Chang and Cohen, 1979, Mol. Gen. Genet. 168: 111-115), by using competent cells (see, e.g., Young and Spizizen, 1961, J. Bacteriol. 81: 823-829, or Dubnau and Davidoff-Abelson, 1971, J. Mol. Biol. 56: 209-221), by electroporation (see, e.g., Shigekawa and Dower, 1988, Biotechniques 6: 742-751), or by conjugation (see, e.g., Koehler and Thorne, 1987, J. Bacteriol. 169: 5271-5278). The introduction of DNA into an E. coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios 68: 189-2070, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023 and Yelton et al., 1984, Proc. Natl. Acad. Sci. USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising: (a) cultivating a host cell of the present invention under conditions suitable for the expression of the variant; and (b) recovering the variant. Accordingly, the present invention relates to methods of producing a variant, comprising (a) cultivating a host cell comprising an expression vector or a polynucleotide encoding variant comprising a substitution at one or more positions corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering, under conditions suitable for the expression of the variant; and (b) recovering the variant.

In one aspect, the present invention relates to a method of obtaining an alpha-amylase variant, comprising introducing into a parent alpha-amylase a substitution at one or more positions corresponding to positions selected from the group consisting of 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering and the variant has alpha-amylase activity; and recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid-state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in cat alogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods may include use of specific antibodies, formation of an enzyme product, or disappearance of an enzyme substrate. For example, an enzyme assay may be used to determine the activity of the variant.

The variant may be recovered by methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure variants.

In an alternative aspect, the variant is not recovered, but rather a host cell of the present invention expressing a variant is used as a source of the variant.

### Compositions

The present invention also relates to compositions comprising a variant of the present invention. Accordingly, the present invention relates to compositions comprising a variant comprising a substitution at one or more positions corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering. Preferably, the compositions are enriched in such a variant. The term “enriched” means that the alpha-amylase activity of the composition has been increased, e.g., with an enrichment factor of 1.1.

The composition may comprise a variant as the major enzymatic component, e.g., a mono-component composition. Alternatively, the composition may comprise multiple enzymatic activities, such as an aminopeptidase, amylase, carbohydrase, carboxypeptidase, cat alase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase. The additional enzyme(s) may be produced, for example, by a microorganism belonging to the genus Bacillus, e.g. Bacillus licheniformis and Bacillus subtilis, or the genus Aspergillus, e.g., Aspergillus aculeatus, Aspergillus awamori, Aspergillus foetidus, Aspergillus fumigatus, Aspergillus japonicus, Aspergillus nidulans, Aspergillus niger, or Aspergillus oryzae; Fusarium, e.g., Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, e.g., Humicola insolens or Humicola lanuginosa; or Trichoderma, e.g., Trichoderma harzianum, Trichoderma koningii, Trichoderma longibrachiatum, Trichoderma reesei, or Trichoderma viride or any other host cell herein described.

The compositions may be prepared in accordance with methods known in the art and may be in the form of a liquid or a dry composition. For instance, the composition may be in the form of a granulate or a microgranulate. The variant may be stabilized in accordance with methods known in the art.

According to the invention, the above alpha-amylase variants may typically be a component in a cleaning composition, such as a detergent composition, e.g., a laundry detergent composition or a dishwashing detergent composition. Especially preferred is a liquid laundry detergent composition.

Such cleaning compositions comprise a cleaning/detergent adjunct, preferably a mixture of components. Typically, the cleaning adjunct will be present in the composition in an amount from 0.001 to 99.9 wt %, more typically from 0.01 to 80 wt % cleaning adjunct.

In another preferred aspect the composition comprises one or more surfactants, which may be non-ionic including semi-polar and/or anionic and/or cationic and/or zwitterionic and/or ampholytic and/or semi-polar nonionic and/or mixtures thereof. The surfactants are typically present at a level of from 0.1% to 60% by weight or from 0.5 to 50 wt % or 1 to 40 wt % of the composition.

### Uses

The present invention is also directed to methods for using the alpha-amylase variants.

The alpha-amylase variants of the invention are useful in detergent compositions, laundry washing, dishwashing and/or cleaning processes.

Method of Use

The present invention also relates to a method for cleaning and/or treating a situs inter alia a surface or fabric. In one aspect, such method comprises the steps of optionally washing and/or rinsing said surface or fabric, contacting said surface or fabric with any consumer product disclosed in this specification then optionally washing and/or rinsing said surface or fabric is disclosed.

As used herein, washing includes but is not limited to, scrubbing, and mechanical agitation. Drying of such surfaces or fabrics may be accomplished by any one of the common means employed either in domestic or industrial settings. Such means include but are not limited to forced air or still air drying at ambient or elevated temperatures at pressures between 5 and 0.01 atmospheres in the presence or absence of electromagnetic radiation, including sunlight, infrared, ultraviolet and microwave irradiation. In one aspect, said drying may be accomplished at temperatures above ambient by employing an iron wherein, for example, said fabric may be in direct contact with said iron for relatively short or even extended periods of time and wherein pressure may be exerted beyond that otherwise normally present due to gravitational force. In another aspect, said drying may be accomplished at temperatures above ambient by employing a dryer. Apparatus for drying fabric is well known and it is frequently referred to as a clothes dryer. In addition to clothes such appliances are used to dry many other items including towels, sheets, pillowcases, diapers and so forth and such equipment has been accepted as a standard convenience in many nations of the world substantially replacing the use of clothes lines for drying of fabric. Most dryers in use today use heated air which is passed over and or through the fabric as it is tumbled within the dryer. The air may be heated, for example, either electronically, via gas flame, or even with microwave radiation. Such air may be heated from about 15° C. to about 400° C., from about 25° C. to about 200° C., from about 35° C. to about 100° C., or even from about 40° C. to about 85° C. and used in the dryer to dry a surface and/or a fabric. As will be appreciated by one skilled in the art, the cleaning compositions of the present invention are ideally suited for use in laundry applications. Accordingly, the present invention includes a method for laundering a fabric. The method comprises the steps of contacting a fabric to be laundered with a said cleaning laundry solution comprising at least one embodiment of Applicants' cleaning composition, cleaning additive or mixture thereof. The fabric may comprise most any fabric capable of being laundered in normal consumer or institutional use conditions. The solution preferably has a pH of from about 4.0 to about 12.0. The compositions may be employed at concentrations of from about 500 ppm to about 15,000 ppm in solution. The water temperatures typically range from about 5° C. to about 90° C. The water to fabric ratio is typically from about 1:1 to about 30:1.

The invention is further described in the following paragraphs.

1. An alpha-amylase variant of a parent alpha-amylase having alpha-amylase activity comprising a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering; and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent polypeptide.
2. The alpha-amylase variant according to paragraph 1, wherein the variant comprises substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering; and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent polypeptide of SEQ ID NO: 1.
3. The alpha-amylase variant according to paragraph 1, wherein the variant comprises substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering; and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent polypeptide of SEQ ID NO: 2.
4. The alpha-amylase variant according to any one of the preceding paragraphs, wherein the variant has an improved property relative to the parent polypeptide, wherein the improved property is selected from the group consisting of increased cat alytic efficiency, increased cat alytic rate, increased chemical stability, increased oxidation stability, increased pH activity, increased pH stability, increased specific activity, increased stability under storage conditions, increased substrate binding, increased substrate cleavage, increased substrate specificity, increased substrate stability, increased surface properties, increased thermal activity, and increased thermostability.
5. The alpha-amylase variant according to any one of the preceding paragraphs, wherein said improved property relative to said parent polypeptide is increased specific activity, particularly increased specific activity in Model A detergent composition.
6. The alpha-amylase variant according to any one of the preceding paragraphs, wherein said improved property is increased specific activity measured as an Improvement Factor (IF) of >1.0 when compared to said parent polypeptide having alpha-amylase activity.
7. The alpha-amylase variant according to any one of the preceding paragraphs, wherein said variant comprises a substitution at a position corresponding to positions 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering; and wherein said alpha-amylase variant has an increased specific activity measured as Improvement Factor (IF) of ≥1.1.
8. The alpha-amylase variant according to any one of the preceding paragraphs, wherein the different amino acid residue is selected from the group consisting of A, C, D, E, F, G, H, I, K, L, M, N, P, Q, R, S, T, V, W, and Y, with the proviso that the different amino acid residue is different from the naturally-occurring amino acid residue.
9. The alpha-amylase variant according to any one of the preceding paragraphs, wherein said variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering, and wherein said alpha-amylase variant has an increased specific activity measured as Improvement Factor (IF) of ≥1.1.
10. The alpha-amylase variant according to any one of the preceding paragraphs, wherein said variant comprises one or more of the following substitutions at a position corresponding to positions N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q; using SEQ ID NO: 1 for numbering, and wherein said alpha-amylase variant has an increased specific activity in Model A detergent composition measured as Improvement Factor (IF) of ≥1.1.
11. The alpha-amylase variant according to any one of the preceding paragraphs, wherein said variant comprises one or more of the following substitutions at a position corresponding to positions N3A; N3C; N3D; N3E; N3F; N3G; N3H; N3L; N3P; N3Q; N3S; N3T; N3V; T5E; G7E; G7F; G7H; G7K; G7L; G7P; G7R; G7S; G7T; G7V; G7W; T8S; H16D; H16R; P18D; P18N; N25T; R26Y; D29N; D30F; R37A; R37N; T40A; T400; T40D; T40E; T40G; T40H; T40I; T40K; T40N; T40V; T40W; A41C; A41D; A41E; A41H; A41I; A41K; A41N; A41R; A41T; A41V; A41Y; W43E; P46A; G50M; G50P; G50Q; G57A; G57S; A60E; A60S; A60V; E68F; Q71F; G73D; G73H; G731; G73L; G73Q; G73S; G73T; G73W; K78S; K78T; T81M; Q841; Q84W; Q84Y; E86K; E86S; S87G; S87L; S87P; H90P; A91Q; A91V; A91W; K93V; V97C; V97H; Q98E; Q98F; Q98G; Q981; Q98K; Q98L; Q98M; Q98R; Q98T; Q98Y; V103C; V103G; V103S; M105F; G110K; G110R; A111E; A111Q; A113T; T114M; T114V; N116Y; V117F; A119H; A119L; A119Y; V120E; E121I; N125S; N126I; N128E; Q129T; I131Y; S132A; S132D; S132H; S1321; S132K; S132L; S132P; S132R; S132T; S132V; S132Y; G133E; G133L; G133T; G133V; Y135C; Y135D; Y135E; Y135P; I137D; I137N; I137Q; I137W; I137Y; E138S; E138V; A139L; W140A; W140D; W140M; W140N; W140P; W140S; W140T; W140V; T141G; K142E; K142N; F143H; F143I; F143M; F145G; F145H; F145I; F145L; F145S; P146A; P146H; P146N; G147D; G149F; G149K; W159A; H161W; F162T; V165P; W167D; W167G; W167P; Q169V; Q169Y; R171P; R176E; R176Q; R176V; R176Y; I177H; I177P; K179A; K179M; K179V; F180D; F180G; R181D; R181E; R181G; R181M; R181Y; K185V; K185W; A186D; W187T; N195Y; G196D; N197V; N197Y; A204F; A204H; A204L; M208D; P211C; P211M; E216L; R219V; T225D; T225F; T225H; T225K; T225L; T225N; T225R; T225Y; L228V; L230C; L230F; D231G; D231T; D231V; I235A; K242L; Y243D; Y243M; Y243N; R247G; R247P; L250S; L250Y; T251D; H252P; V253F; V253S; R254D; R254F; R254H; R254P; R254T; R254V; R254W; N255C; N255F; N255P; T257A; T257C; T257D; T257E; T257G; T257P; T257Q; T257S; T257W; T257Y; K259P; K259V; K259W; K259Y; E260V; M261D; M261F; M261P; M261R; M261S; M261W; F262A; F262C; F262E; F262H; F262I; F262K; F262L; F262N; F262P; F262Q; F262R; F262S; F262T; F262V; F262W; A263E; A263H; A263W; A265Q; L272W; G273Q; L279I; N280P; K281H; K281I; K281S; N283P; W284A; W284D; W284E; W284G; W284I; W284K; W284M; W284N; W284P; W284Q; W284S; W284T; W284V; W284Y; S2871; S287K; S287L; S287P; S287R; S287T; S287V; S287Y; P292L; P292M; P292Y; H294N; H294S; N296T; Y298D; Y298E; N299T; A300G; S303A; S303C; S303D; S303E; S303F; S303G; S303H; S303L; S303M; S303N; S303Q; S303T; S303V; S303Y; G305E; Y307S; A310D; A310N; K311A; K311C; L313Q; N314P; N314S; G315E; G315M; G315V; Q319C; Q319E; K320S; K320W; H321F; H321K; H321L; H321R; H321T; H321V; H321W; P322C; P322E; P322H; P322T; P322Y; M323A; M323C; M323D; M323E; M323G; M323H; M323L; M323N; M323P; M323R; M323S; M323Y; H324A; H324I; H324P; H324T; H324W; H324Y; S334V; G337V; L340M; S342A; S342G; S342T; V344A; V344C; V344I; Q345N; E346A; W347A; W347E; W347F; W347G; W347H; W347K; W347N; W347P; W347R; P350A; P350C; P350N; P350T; R359C; R359F; R359G; R359M; R359W; E360D; E360H; E360P; E360T; E360W; Q361D; Q361H; Q3611; Q361K; Q361L; Q361M; Q361N; Q361P; Q361R; Q361S; Q361T; Q361V; Q361W; Q361Y; G362M; Y363A; Y363E; Y363F; Y363G; Y3631; Y363L; Y363Q; Y363S; Y363T; Y363V; Y363W; P364C; Y368C; Y368Q; Y368R; Y368T; Y372C; Y372G; Y372N; Y372T; P375Q; P375T; P380F; M3821; K383C; K383L; K383P; K383T; A384D; A384N; K385N; K385V; D387C; D387P; D387R; D387W; P388I; E391M; R393K; R393M; N395C; N395D; N395K; N395V; N395W; F396E; F396G; F396T; A397V; Y398E; Q401H; H402S; D403A; D403E; D403T; F405C; F405N; F405S; F405T; D406V; H407C; H407Q; H407T; I41C H; I41C M; I41C R; I41C W; R415C; R415L; R415M; R415Y; E416D; E416F; E416L; E416N; N418A; N418P; N418V; H421A; H421G; H421L; H421P; H421Q; H421Y; P422D; P422G; P422I; P422N; P422Q; P422S; N423G; N423H; N423I; N423L; N423M; N423Q; N423V; N423W; N423Y; G433A; G433S; P434I; P434L; G435C; G435L; G435M; G435T; G435V; K438A; K438I; K438Q; K438S; K438V; W439A; W439D; W439I; W439K; W439L; W439N; W439S; W439T; W439V; W439Y; K446A; K446F; A447D; A447Y; Q449T; V450D; V450N; H452L; T455C; T455M; T455V; N457D; N457E; N457I; N457L; N457V; K458D; K458L; G460M; T461F; T461I; T461Q; T461V; V462C; N465A; N465C; N465E; N465G; N465H; N465L; N465P; N465T; A466F; A466I; A466Y; D4671; W469A; W469E; W469H; W469L; W469N; W469Q; W469R; W469Y; N471A; N471D; N471M; N471W; S473N; N475A; N475C; N475D; N475E; N475F; N475G; N475H; N475I; N475L; N475M; N475P; N475Q; N475S; N475T; N475V; N475W; G476F; G476L; G477A; G477H; G477Q; G477S; G477W; S478A; S478F; and S478Q, using SEQ ID NO: 1 for numbering, and wherein said alpha-amylase variant has an increased specific activity in Model A detergent composition measured as Improvement Factor (IF) of ≥1.2.
12. The alpha-amylase variant according to any one of the preceding paragraphs, wherein said variant comprises one or more of the following substitutions at a position corresponding to positions N3A; N3C; N3D; N3E; N3F; N3G; N3H; N3L; N3P; N3Q; N3V; T5E; G7E; G7F; G7H; G7L; G7R; G7S; G7T; G7V; G7W; H16D; R26Y; D29N; D30F; R37A; R37N; T40A; T400; T40D; T40E; T40G; T40H; T40I; T40N; T40V; T40W; A41C; A41D; A41E; A41H; A41I; A41K; A41N; A41R; A41Y; P46A; G50Q; G57A; G57S; A60S; A60V; E68F; G73D; G73H; G731; G73L; G73Q; G73S; G73T; G73W; K78T; T81M; Q841; Q84W; E86S; S87G; H90P; Q98F; Q981; Q98K; Q98L; Q98M; Q98Y; V103C; V103G; G110K; G110R; A111E; A111Q; A119L; A119Y; V120E; N125S; N128E; I131Y; S132A; S132D; S132H; S1321; S132K; S132L; S132R; S132V; S132Y; G133E; Y135C; Y135D; Y135E; Y135P; I137N; I137W; E138S; E138V; A139L; W140A; W140D; W140M; W140N; W140P; W140S; W140T; K142E; F143H; F143I; F145I; P146A; P146H; P146N; G147D; G149K; H161W; V165P; W167D; W167P; Q169V; R171P; R176E; R176Q; R176Y; I177H; K179A; K179V; R181E; R181G; R181M; R181Y; A186D; W187T; G196D; N197V; N197Y; A204H; A204L; P211C; P211M; T225D; T225F; T225H; T225K; T225L; T225N; T225R; T225Y; L230C; L230F; D231V; Y243D; Y243N; R247G; R247P; L250S; H252P; V253F; R254T; R254V; R254W; N255P; T257A; T257C; T257D; T257E; T257G; T257W; K259P; K259V; K259W; E260V; M261D; M261F; M261P; M261R; M261S; M261W; F262A; F262C; F262E; F262H; F262I; F262K; F262L; F262N; F262P; F262Q; F262R; F262S; F262T; F262V; F262W; A263E; A263H; A263W; A265Q; L279I; K281I; N283P; W284A; W284D; W284E; W284G; W2841; W284K; W284M; W284N; W284P; W284Q; W284S; W284T; W284V; W284Y; S287K; S287L; S287R; S287T; S287V; S287Y; H294S; N296T; Y298D; N299T; S303A; S303C; S303D; S303E; S303F; S303G; S303H; S303L; S303M; S303N; S303Q; S303T; S303V; S303Y; G305E; Y307S; A310N; K311A; K311C; L313Q; N314P; N314S; G315E; G315V; Q319E; K320W; H321F; H321K; H321L; H321V; P322C; P322E; P322H; P322T; P322Y; M323A; M323C; M323D; M323E; M323G; M323H; M323L; M323N; M323P; M323R; M323S; M323Y; H324A; H324I; H324P; H324T; H324W; H324Y; G337V; L340M; S342G; V344C; E346A; W347E; W347F; W347G; W347H; W347K; W347N; W347P; W347R; P350C; P350N; P350T; R359G; R359W; E360D; E360T; E360W; Q361D; Q361H; Q361I; Q361K; Q361L; Q361M; Q361N; Q361P; Q361R; Q361T; Q361V; Q361W; Q361Y; G362M; Y363A; Y363F; Y363G; Y3631; Y363Q; Y363S; Y363T; Y363V; Y363W; P364C; Y368C; Y368Q; Y368R; Y372C; Y372G; P375Q; P375T; P380F; K383C; K383T; A384D; K385N; K385V; D387C; D387P; D387R; E391M; R393K; R393M; N395C; N395D; N395W; F396E; F396G; F396T; Y398E; H402S; D403A; D403T; F405C; F405N; F405S; F405T; D406V; H407C; H407Q; H407T; I41C H; I41C M; I41C W; R415C; R415L; R415M; R415Y; E416F; E416L; E416N; N418A; N418P; H421A; H421G; H421L; H421P; H421Q; H421Y; P422D; P422G; P422N; P422S; N423G; N423I; N423L; N423M; N423Q; N423V; N423W; N423Y; G433S; P434I; P434L; G435M; G435T; G435V; K4381; K438Q; K438S; K438V; W439A; W439D; W439I; W439K; W439L; W439N; W439S; W439T; W439V; K446A; K446F; A447D; A447Y; H452L; T455V; N457D; N457E; N457I; N457L; N457V; K458D; K458L; G460M; T461F; T461I; T461Q; T461V; V462C; N465A; N465E; N465G; N465L; A466F; A466I; A466Y; D4671; W469A; W469E; W469H; W469L; W469Q; W469R; W469Y; N471A; N471D; N471M; N475A; N475C; N475D; N475E; N475F; N475G; N475H; N475I; N475L; N475M; N475P; N475Q; N475S; N475T; N475V; N475W; G476L; G477A; G477H; G477Q; G477S; G477W; S478A; and S478F; using SEQ ID NO: 1 for numbering, and wherein said alpha-amylase variant has an increased specific activity in Model A detergent composition measured as Improvement Factor (IF) of ≥1.3.
13. The alpha-amylase variant according to any one of the preceding paragraphs, wherein said variant comprises one or more of the following substitutions at a position corresponding to positions N3A; N3C; N3D; N3E; N3F; N3G; N3H; N3L; N3V; G7F; G7H; G7R; G7V; G7W; R26Y; D29N; R37A; R37N; T400; T40D; T40E; T40G; T40I; T40N; T40V; A41C; A41D; A41E; A41K; A41N; A41R; A41Y; G73D; G73H; G731; G73L; G73Q; G73S; G73T; G73W; K78T; Q841; Q84W; S87G; Q981; Q98L; V103C; G110K; A111Q; A119Y; N125S; N128E; S132D; S1321; 5132L; S132R; Y135C; Y135E; I137W; E138S; E138V; A139L; W140A; W140D; W140M; W140N; W140P; W140S; W140T; K142E; F143I; F145I; P146H; G149K; H161W; V165P; W167D; W167P; Q169V; R176Q; I177H; P211C; T225F; T225H; T225K; T225R; T225Y; L230C; Y243D; R247G; V253F; N255P; T257A; T257D; T257E; K259P; K259V; M261D; M261R; M261W; F262A; F262C; F262E; F262H; F262I; F262K; F262L; F262N; F262P; F262Q; F262R; F262S; F262W; A263H; L279I; N283P; W284D; W284E; W284I; W284K; W284M; W284N; W284P; W284Q; W284S; W284V; S287K; S287L; S287R; S287Y; N296T; S303A; S303C; S303D; S303E; S303F; S303G; S303H; S303M; S303N; S303Q; S303T; S303V; S303Y; G305E; K311C; L313Q; N314P; G315V; H321K; H321L; H321V; P322C; P322E; P322H; P322T; P322Y; M323A; M323D; M323E; M323G; M323L; M323P; M323S; M323Y; H324A; H324I; H324P; H324W; L340M; S342G; E346A; W347F; W347K; W347N; W347P; W347R; R359W; E360W; Q361D; Q361H; Q361I; Q361K; Q361L; Q361M; Q361P; Q361R; Q361V; Q361W; Q361Y; G362M; Y363A; Y363G; Y363T; Y363V; Y363W; P364C; Y368C; Y368Q; Y372C; Y372G; K383T; A384D; D387C; D387P; E391M; R393K; R393M; N395D; N395W; F396E; F396T; D403A; F405C; F405N; F405S; D406V; H407C; H407T; I41C H; I41C M; I41C W; R415C; R415L; R415M; R415Y; N418P; H421A; H421L; H421P; H421Q; P422D; P422G; N423L; N423V; N423W; N423Y; G433S; G435M; K438V; W439A; W439I; W439K; W439S; W439T; W439V; K446A; A447Y; H452L; T455V; N457E; N457I; N457V; K458D; T461F; T461Q; N465A; N465E; N465G; A466I; D467I; W469A; W469E; W469Y; N471A; N471D; N471M; N475A; N475C; N475D; N475E; N475F; N475G; N475H; N475L; N475M; N475P; N475Q; N475S; N475T; N475V; N475W; G477A; G477H; G477Q; G477S; G477W; S478A; using SEQ ID NO: 1 for numbering, and wherein said alpha-amylase variant has an increased specific activity in Model A detergent composition measured as Improvement Factor (IF) of ≥1.4.
14. The alpha-amylase variant according to any one of the preceding paragraphs, wherein said variant comprises one or more of the following substitutions at a position corresponding to positions N3A; N3C; N3D; N3E; N3G; N3L; G7F; R26Y; R37A; R37N; T400; T40D; T40G; T40I; T40N; T40V; A41C; A41D; A41K; A41N; A41R; G73D; G731; G73L; G73Q; G73S; G73T; G73W; K78T; Q841; G110K; A111Q; N128E; Y135E; A139L; W140A; W140M; K142E; F145I; G149K; V165P; W167P; R176Q; I177H; T225F; T225H; T225K; T225Y; L230C; R247G; T257D; T257E; M261W; F262A; F262E; F262K; F262N; F262P; F262Q; F262R; F262S; F262W; N283P; W284D; W284P; W284S; W284V; S287Y; S303A; S303C; S303D; S303E; S303F; S303G; S303H; S303M; S303N; S303Q; S303T; S303V; S303Y; L313Q; N314P; H321K; H321L; H321V; P322C; P322H; P322Y; M323A; M323D; M323Y; H324P; H324W; L340M; S342G; E346A; W347N; W347P; W347R; R359W; E360W; Q361K; Q361L; Q361M; Q361R; Q361W; Q361Y; Y363G; Y363V; Y363W; Y368C; Y372C; K383T; D387P; E391M; R393K; R393M; N395D; N395W; D403A; F405C; F405S; D406V; H407C; H407T; I41C H; R415C; R415L; R415M; R415Y; N418P; H421A; H421L; H421P; P422D; N423L; N423V; G435M; W439A; K446A; H452L; T455V; N457I; K458D; N465E; N471A; N471D; N471M; N475A; N475C; N475D; N475E; N475F; N475G; N475H; N475L; N475P; N475Q; N475V; N475W; G477A; G477H; G477Q; and G477S; using SEQ ID NO: 1 for numbering, and wherein said alpha-amylase variant has an increased specific activity in Model A detergent composition measured as Improvement Factor (IF) of ≥1.5
15. The alpha-amylase variant according to any one of the preceding paragraphs, wherein said variant comprises one or more of the following substitutions at a position corresponding to positions R37A; R176Q; T225Y; F262P; N283P; S303C; S303D; S303G; S303M; S303N; S303T; S303V; S303Y Y368C; N395D; H407T; I41C H; R415C; H421A; G435M; N475C; G477A; and G477H; using SEQ ID NO: 1 for numbering, and wherein said alpha-amylase variant has an increased specific activity in Model A detergent composition measured as Improvement Factor (IF) of ≥2.0.
16. The alpha-amylase variant according to any one of the preceding paragraphs, wherein said variant comprises one or more of the following substitutions at a position corresponding to positions S303C; S303T; Y368C; and H421A; using SEQ ID NO: 1 for numbering, and wherein said alpha-amylase variant has an increased specific activity in Model A detergent composition measured as Improvement Factor (IF) of ≥2.5.
17. The alpha-amylase variant according to any one of the preceding paragraphs, further comprising deletion at two or more positions corresponding to positions R181, G182, D183, and G184, using SEQ ID NO: 1 for numbering.
18. The alpha-amylase variant according to paragraph 17, wherein the deletion is selected from the group consisting of R181*+G182*, R181*+D183*, R181*+G184*, G182*+D183*, G182*+G184*, or D183*+G184*, preferably D183*+G184*.
19. The alpha-amylase variant according to any one of the preceding paragraphs, wherein the parent polypeptide has at least 60%, e.g., at least 62%, at least 63%, at least 65%, at least 70%, at least 75%, at least 80%, at least 85%, at least 90%, at least 95%, at least 96%, at least 97%, at least 98%, at least 99% but less than 100% sequence identity to the polypeptide of SEQ ID NO: 1, 2, 3, 4, 5, 6, 7, 8, 9, 10, 11, 12, 13, 14, 15, 16 or 17.
20. The alpha-amylase variant according to any one of the preceding paragraphs, wherein said parent polypeptide comprises or consists of the polypeptide of SEQ ID NO: 1 or SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 or SEQ ID NO: 17.
21. The alpha-amylase variant according to any one of the preceding paragraphs, which has at least 60%, e.g., at least 65%, at least 70%, at least 75%, at least 80%, at least 85%, at least 90%, at least 95% identity, at least 96%, at least 97%, at least 98%, or at least 99%, but less than 100%, sequence identity to the amino acid sequence of the parent polypeptide. 22. The alpha-amylase variant according to any one of the preceding paragraphs, wherein said variant comprises a substitution at 1, 2, 3, 4, 5, 6, 7, 8, 9, or 10 positions.
23. A polynucleotide encoding the variant according to any one of the preceding paragraphs 1-22.
24. A nucleic acid construct comprising the polynucleotide according to paragraph 23.
25. An expression vector comprising the polynucleotide according to paragraph 23.
26. A host cell comprising the polynucleotide according to paragraph 23, the nucleic acid construct according to paragraph 24, or the expression vector according to paragraph 25.
27. A composition comprising the variant according to any one of paragraph 1 to 22 and at least one additional active component.
28. The composition according to paragraph 27, wherein the at least one additional active component is an enzyme.
29. The composition according to paragraph 28, wherein said enzyme is selected from the group consisting of a protease, lipase, cellulase, pectate lyase and mannanase.
30. The composition according to any one of paragraph 27 to 29, wherein said composition is a detergent composition, such as a liquid or powder detergent composition.
31. The composition according to any one of paragraph 27 to 30, wherein said composition is a liquid laundry or liquid dish wash composition, such as an Automatic Dish Wash (ADW) liquid detergent composition, or a powder laundry, such as a soap bar, or powder dish wash composition, such as an ADW unit dose detergent composition and such as a Hand Dish Wash (HDW) detergent composition.
32. A method of obtaining an alpha-amylase variant of a parent alpha-amylase comprising the steps of:
a) introducing a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering;
b) optionally introducing a deletion at two or more positions in the parent alpha-amylase said positions R181, G182, D183, and G184, using SEQ ID NO: 1 for numbering;
and said method thereby providing an alpha-amylase variant of said parent alpha-amylase, wherein said variant has at least 60%, such as at least 65%, such as at least 70%, such as at least 75%, such as at least 80%, such as at least 85%, such as at least 90%, such as at least 95%, such as at least 97%, such as at least 99%, but less than 100%, sequence identity to the amino acid sequence to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent.
33. Use of a variant according to any one of paragraphs 1 to 20 in a cleaning process such as laundry or hard surface cleaning including dish wash and industrial cleaning.

### EXAMPLES

### Example 1: Generation of Variants According to the Invention

Enzyme: SP722: SEQ ID NO: 1, available from Novozymes, and disclosed in WO 95/26397. Amylase variants of SEQ ID NO: 1 (SP722) were prepared by standard procedures, in brief: Introducing random and/or site-directed mutations into the gene, transforming Bacillus subtilis host cells with the mutated genes, fermenting the transformed host cells (e.g. as described in Example 1 of WO 2004/111220). The reference amylase or parent polypeptide of SEQ ID NO: 1 was produced recombinantly in Bacillus subtilis in a similar manner.

A library of variants were generated with a substitution in different positions selected from the following positions: N3A, N3C, N3D, N3E, N3F, N3G, N3H, N3L, N3P, N3Q, N3S, N3T, N3V, T5E, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T400, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V4620, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q, using SEQ ID NO: 1 for numbering.

### Example 2: Specific Activity of Variants

In order to determine whether the variants generated as described in Example 1 have a maintained or even improved activity, the variants were evaluated by the micro-swatch assay. The following detergent composition was prepared

TABLE 1
Model detergent A (percentages given in w/w)

| | Content of compound | % active component |
|---|---|---|
| Compound | (% w/w) | (% w/w) |
| LAS | 12.00 | 11.60 |
| AEOS, SLES | 17.63 | 4.90 |
| soy fatty acid | 2.75 | 2.48 |
| coco fatty acid | 2.75 | 2.80 |
| AEO | 11.00 | 11.00 |
| Sodium hydroxide | 1.75 | 1.80 |
| Ethanol/propan-2-ol | 3.00 | 2.70/0.30 |
| MPG | 6.00 | 6.00 |
| Glycerol | 1.71 | 1.70 |
| TEA | 3.33 | 3.30 |
| Sodium formate | 1.00 | 1.00 |
| Sodium citrate | 2.00 | 2.00 |
| DTMPA | 0.48 | 0.20 |
| PCA | 0.46 | 0.18 |
| Phenoxy ethanol | 0.50 | 0.50 |
| H₂O, ion exchanged | 33.64 | 33.64 |

### Preparation of Model A Detergent (0.33%):

4:1 Molar Ratio of CaCl₂) and MgCl2 Stock Solution with 6000 dH (Water Hardness). 125.8 g of CaCl2.2H2O was weighed into 1-liter bottle and to this 500 ml of type I water was added and stirred well. To this 43.8 g of MgCl2.6H2O was weighed and added and dissolved well and the final volume was made up to 1000 ml with type I water.
0.535 M Solution of NaHCO₃: 44.9 g of Sodium Hydrogen carbonate was dissolved in 100 ml of type I water.
Model A Detergent with a Water Hardness of 15 (15° dH): 3.335 g of Model A detergent was weighed and transferred into 1 litre bottle and to this 865 ml of type I water was added and mixed well. To this 7.5 ml of 0.535M NaHCO₃ was added, mixed well and made up the volume to 1 liter with type 1 water. To adjust the water hardness to 15° dH, 2.5 ml of 4:1 molar ratio of CaCl₂.2H₂O and MgCl₂.6H₂O stock solution with 6000° dH was added and the mixture was stirred for 15 min.

### Experimental Procedure

### Preparation of the Mother Plates:

Colonies were picked from the transformed plate by colony picker (KBiosystems) and inoculated in 96-well culture plate comprising TBGly media for growth. The cultures were grown for 3 days at 37° C. and the supernatant was recovered from the plates by centrifugation.

### Preparation of the Substrate Plate:

5-meter swatches—CS-27 were obtained from Center for Testmaterials (CFT, The Netherlands) and were cut into 6 mm micro swatches using swatch puncher. One swatch was placed in each well of 96-well Nunc plates to which 180 μl of Model A detergent (0.33%) was added. The culture supernatants were diluted to 500λ with 100 mM MOPS, pH 7 with 0.1 mM CaCl₂), 0.01% Triton-X. To the plates containing the swatches and Model A detergent, 20 ul of diluted supernatants was added and incubated at 25° C., 800 RPM for 10 min. 40 ul of the reaction mixture was transferred to another 96-well plate and in order to digest all the released oligosaccharides to glucose, 10 ul of 0.1 mg/ml of Amyloglucosidase (AMG) was added and incubated for 5 min at room temperature. AMG was purified from commercial product AMG 300L (Novozymes). The reducing ends were detected by p-hydroxybenzoic acid hydrazide (PAHBAH) method (Lever M Anal Biochem. 81:21-7). To prepare 1.5% PAHBAH reagent, 1.5 g of p-hydroxybenzoic acid hydrazide (PAHBAH) was dissolved in the buffer containing 1.0 M Na2CO3, 0.17 M potassium sodium tartrate and 5 mM Bi(NO3)3.5H2O. 50 ul AMG digest was added 50 ul of 1.5% PAHBAH reagent and heated at 95° C. for 10 mins. Absorbance of the resultant mixture was measured at 405 nm which is proportional to the enzyme activity. The concentration of the expressed enzyme was determined by ELISA using specific antibodies. The specific activity was calculated by taking the ratio of the specific activity by concentration and the hits were identified as anything higher than the specific activity of the parent alpha-amylase. The reference alpha-amylase or the parent alpha-amylase is of SEQ ID NO: 1. Improvement Factor (IF) of reference alpha-amylase or the parent alpha-amylase is IF=1.0.

The Improvement Factor (IF) was calculated as:

Improvement Factor (IF)=[Specific Activity of variant]/[Specific Activity of parent alpha-amylase]

TABLE 2
Improvement Factor (IF) of the variants as culture supernatant according
to the invention obtained by the method described above are the
following (amino acid substitutions refer to SEQ ID NO: 1):

| Substitu- | | Substitu- | | Substitu- | | Substitu- | |
|---|---|---|---|---|---|---|---|
| tion | IF | tion | IF | tion | IF | tion | IF |
| N3A | 1.5 | W140V | 1.2 | S287K | 1.4 | D387W | 1.2 |
| N3C | 1.9 | T141G | 1.2 | S287L | 1.4 | P388I | 1.2 |
| N3D | 1.7 | K142E | 1.8 | S287P | 1.2 | E391M | 1.6 |
| N3E | 1.5 | K142N | 1.2 | S287R | 1.4 | R393K | 1.5 |
| N3F | 1.4 | F143H | 1.3 | S287T | 1.3 | R393M | 1.5 |
| N3G | 1.6 | F143I | 1.4 | S287V | 1.3 | R393R | 1.3 |
| N3H | 1.4 | F143M | 1.2 | S287Y | 1.6 | N395C | 1.3 |
| N3L | 1.5 | F145G | 1.2 | P292L | 1.2 | N395D | 2.0 |
| N3P | 1.3 | F145H | 1.2 | P292M | 1.2 | N395K | 1.2 |
| N3Q | 1.3 | F145I | 1.5 | P292Y | 1.2 | N395V | 1.2 |
| N3S | 1.2 | F145L | 1.2 | H294N | 1.2 | N395W | 1.9 |
| N3T | 1.2 | F145S | 1.2 | H294S | 1.3 | F396E | 1.4 |
| N3V | 1.4 | P146A | 1.3 | N296T | 1.4 | F396G | 1.3 |
| T5E | 1.3 | P146H | 1.4 | Y298D | 1.3 | F396T | 1.4 |
| G7E | 1.3 | P146N | 1.3 | Y298E | 1.2 | A397V | 1.2 |
| G7F | 1.5 | G147D | 1.3 | N299T | 1.3 | Y398E | 1.3 |
| G7H | 1.4 | G149F | 1.2 | A300G | 1.2 | Q401H | 1.2 |
| G7K | 1.2 | G149K | 1.5 | S303A | 1.9 | H402S | 1.3 |
| G7L | 1.3 | W159A | 1.2 | S303C | 3.2 | D403A | 1.5 |
| G7P | 1.2 | W159E | 1.1 | S303D | 2 | D403E | 1.2 |
| G7R | 1.4 | H161W | 1.4 | S303E | 1.8 | D403T | 1.3 |
| G7S | 1.3 | F162T | 1.2 | S303F | 1.9 | F405C | 1.5 |
| G7T | 1.3 | V165P | 1.5 | S303G | 2 | F405N | 1.4 |
| G7V | 1.4 | W167D | 1.4 | S303H | 1.9 | F405S | 1.5 |
| G7W | 1.4 | W167G | 1.2 | S303L | 1.3 | F405T | 1.3 |
| T8S | 1.2 | W167P | 1.9 | S303M | 2.1 | D406V | 1.6 |
| H16D | 1.3 | Q169L | 1.1 | S303N | 2.1 | H407C | 1.5 |
| H16R | 1.2 | Q169P | 1.1 | S303Q | 1.9 | H407Q | 1.3 |
| P18D | 1.2 | Q169V | 1.4 | S303S | 1.8 | H407T | 2.0 |
| P18L | 1.1 | Q169Y | 1.2 | S303T | 2.7 | I410H | 2.0 |
| P18N | 1.2 | R171P | 1.3 | S303V | 2.3 | I410M | 1.4 |
| N25T | 1.2 | R176E | 1.3 | S303Y | 2.4 | I410R | 1.2 |
| R26Y | 1.7 | R176Q | 2.1 | G305E | 1.4 | I410W | 1.4 |
| D29N | 1.4 | R176V | 1.2 | Y307S | 1.3 | R415C | 2.1 |
| D30F | 1.3 | R176Y | 1.3 | A310D | 1.2 | R415L | 1.6 |
| R35P | 1.1 | I177H | 1.8 | A310N | 1.3 | R415M | 1.7 |
| R37A | 2.1 | I177P | 1.2 | K311A | 1.3 | R415Y | 1.7 |
| R37N | 1.8 | K179A | 1.3 | K311C | 1.4 | E416D | 1.2 |
| T40A | 1.3 | K179M | 1.2 | L313Q | 1.8 | E416F | 1.3 |
| T40C | 1.7 | K179V | 1.3 | N314P | 1.5 | E416L | 1.3 |
| T40D | 1.5 | F180D | 1.2 | N314S | 1.3 | E416N | 1.3 |
| T40E | 1.4 | F180G | 1.2 | G315E | 1.3 | N418A | 1.3 |
| T40G | 1.5 | R181D | 1.2 | G315M | 1.2 | N418P | 1.5 |
| T40H | 1.3 | R181E | 1.3 | G315V | 1.4 | N418V | 1.2 |
| T40I | 1.6 | R181G | 1.3 | Q319C | 1.2 | H421A | 2.6 |
| T40K | 1.2 | R181M | 1.3 | Q319E | 1.3 | H421G | 1.3 |
| T40N | 1.5 | R181Y | 1.3 | K320S | 1.2 | H421L | 1.5 |
| T40V | 1.5 | K185T | 1.1 | K320W | 1.3 | H421P | 1.9 |
| T40W | 1.3 | K185V | 1.2 | H321F | 1.3 | H421Q | 1.4 |
| A41C | 1.5 | K185W | 1.2 | H321K | 1.5 | H421Y | 1.3 |
| A41D | 1.6 | A186D | 1.3 | H321L | 1.5 | P422D | 1.7 |
| A41E | 1.4 | W187T | 1.3 | H321R | 1.2 | P422G | 1.4 |
| A41H | 1.3 | N195Y | 1.2 | H321T | 1.2 | P422I | 1.2 |
| A41I | 1.3 | G196D | 1.3 | H321V | 1.6 | P422N | 1.3 |
| A41K | 1.5 | N197V | 1.3 | H321W | 1.2 | P422Q | 1.2 |
| A41N | 1.7 | N197Y | 1.3 | P322C | 1.5 | P422S | 1.3 |
| A41R | 1.6 | A204F | 1.2 | P322E | 1.4 | N423G | 1.3 |
| A41T | 1.2 | A204H | 1.3 | P322H | 1.6 | N423H | 1.2 |
| A41V | 1.2 | A204L | 1.3 | P322T | 1.4 | N423I | 1.3 |
| A41Y | 1.4 | V206N | 1.1 | P322Y | 1.6 | N423L | 1.5 |
| W43E | 1.2 | M208D | 1.2 | M323A | 1.5 | N423M | 1.3 |
| P46A | 1.3 | P211C | 1.4 | M323C | 1.3 | N423P | 1.1 |
| G50M | 1.2 | P211M | 1.3 | M323D | 1.6 | N423Q | 1.3 |
| G50P | 1.2 | E216L | 1.2 | M323E | 1.4 | N423V | 1.5 |
| G50Q | 1.3 | R218Q | 1.1 | M323G | 1.4 | N423W | 1.4 |
| G57A | 1.3 | R219V | 1.2 | M323H | 1.3 | N423Y | 1.4 |
| G57S | 1.3 | T225D | 1.3 | M323L | 1.4 | G433A | 1.2 |
| A60E | 1.2 | T225F | 1.9 | M323N | 1.3 | G433N | 1.1 |
| A60S | 1.3 | T225H | 1.7 | M323P | 1.4 | G433S | 1.4 |
| A60V | 1.3 | T225K | 1.5 | M323R | 1.3 | P434I | 1.3 |
| E68F | 1.3 | T225L | 1.3 | M323S | 1.4 | P434L | 1.3 |
| E68Y | 1.1 | T225N | 1.3 | M323T | 1.1 | G435C | 1.2 |
| Q71F | 1.2 | T225R | 1.4 | M323W | 1.1 | G435L | 1.2 |
| G73D | 1.6 | T225Y | 2 | M323Y | 1.6 | G435M | 2 |
| G73H | 1.4 | L228V | 1.2 | H324A | 1.4 | G435T | 1.3 |
| G73I | 1.5 | L230C | 1.5 | H324I | 1.4 | G435V | 1.3 |
| G73L | 1.9 | L230F | 1.3 | H324P | 1.6 | K438A | 1.2 |
| G73Q | 1.5 | D231G | 1.2 | H324T | 1.3 | K438I | 1.3 |
| G73S | 1.5 | D231T | 1.2 | H324W | 1.5 | K438Q | 1.3 |
| G73T | 1.5 | D231V | 1.3 | H324Y | 1.3 | K438S | 1.3 |
| G73W | 1.7 | I235A | 1.2 | S334V | 1.2 | K438V | 1.4 |
| T74F | 1.1 | K242L | 1.2 | G337V | 1.3 | W439A | 1.5 |
| K78S | 1.2 | Y243D | 1.4 | S339S | 1.2 | W439D | 1.3 |
| K78T | 1.7 | Y243M | 1.2 | L340M | 1.7 | W439I | 1.4 |
| T81M | 1.3 | Y243N | 1.3 | S342A | 1.2 | W439K | 1.4 |
| Q84I | 1.7 | R247G | 1.5 | S342G | 1.5 | W439L | 1.3 |
| Q84W | 1.4 | R247P | 1.3 | S342T | 1.2 | W439N | 1.3 |
| Q84Y | 1.2 | R247S | 1.1 | V344A | 1.2 | W439S | 1.4 |
| E86K | 1.2 | L250S | 1.3 | V344C | 1.3 | W439T | 1.4 |
| E86S | 1.3 | L250Y | 1.2 | V344I | 1.2 | W439V | 1.4 |
| S87G | 1.4 | T251D | 1.2 | Q345N | 1.2 | W439Y | 1.2 |
| S87L | 1.2 | H252P | 1.3 | E346A | 1.8 | K446A | 1.8 |
| S87P | 1.2 | V253F | 1.4 | W347A | 1.2 | K446F | 1.3 |
| H90P | 1.3 | V253S | 1.2 | W347E | 1.3 | A447D | 1.3 |
| A91Q | 1.2 | R254D | 1.2 | W347F | 1.4 | A447Y | 1.4 |
| A91V | 1.2 | R254F | 1.2 | W347G | 1.3 | Q449T | 1.2 |
| A91W | 1.2 | R254H | 1.2 | W347H | 1.3 | V450D | 1.2 |
| K93V | 1.2 | R254P | 1.2 | W347K | 1.4 | V450N | 1.2 |
| V97C | 1.2 | R254T | 1.3 | W347N | 1.5 | H452L | 1.7 |
| V97H | 1.2 | R254V | 1.3 | W347P | 1.9 | T455C | 1.2 |
| Q98E | 1.2 | R254W | 1.3 | W347R | 1.5 | T455M | 1.2 |
| Q98F | 1.3 | N255C | 1.2 | P350A | 1.2 | T455V | 1.5 |
| Q98G | 1.2 | N255F | 1.2 | P350C | 1.3 | N457D | 1.3 |
| Q98I | 1.4 | N255P | 1.4 | P350N | 1.3 | N457E | 1.4 |
| Q98K | 1.3 | T257A | 1.4 | P350T | 1.3 | N457I | 1.6 |
| Q98L | 1.4 | T257C | 1.3 | R359C | 1.2 | N457L | 1.3 |
| Q98M | 1.3 | T257D | 1.5 | R359F | 1.2 | N457V | 1.4 |
| Q98R | 1.2 | T257E | 1.6 | R359G | 1.3 | K458D | 1.8 |
| Q98T | 1.2 | T257G | 1.3 | R359M | 1.2 | K458L | 1.3 |
| Q98Y | 1.3 | T257P | 1.2 | R359S | 1.1 | G460M | 1.3 |
| V103C | 1.4 | T257Q | 1.2 | R359W | 1.5 | G460V | 1.1 |
| V103G | 1.3 | T257S | 1.2 | E360D | 1.3 | T461F | 1.4 |
| V103S | 1.2 | T257W | 1.3 | E360H | 1.2 | T461I | 1.3 |
| M105F | 1.2 | T257Y | 1.2 | E360P | 1.2 | T461Q | 1.4 |
| G110K | 1.5 | K259P | 1.4 | E360T | 1.3 | T461V | 1.3 |
| G110R | 1.3 | K259V | 1.4 | E360W | 1.5 | V462C | 1.3 |
| A111E | 1.3 | K259W | 1.3 | Q361D | 1.4 | N465A | 1.4 |
| A111Q | 1.6 | K259Y | 1.2 | Q361H | 1.4 | N465C | 1.2 |
| A113T | 1.2 | E260V | 1.3 | Q361I | 1.4 | N465E | 1.5 |
| T114M | 1.2 | M261D | 1.4 | Q361K | 1.6 | N465G | 1.4 |
| T114V | 1.2 | M261F | 1.3 | Q361L | 1.6 | N465H | 1.2 |
| N116C | 1.1 | M261P | 1.3 | Q361M | 1.6 | N465L | 1.3 |
| N116Y | 1.2 | M261R | 1.4 | Q361N | 1.3 | N465P | 1.2 |
| V117F | 1.2 | M261S | 1.3 | Q361P | 1.4 | N465T | 1.2 |
| A119H | 1.2 | M261W | 1.6 | Q361R | 1.5 | A466F | 1.3 |
| A119L | 1.3 | F262A | 1.9 | Q361S | 1.2 | A466I | 1.4 |
| A119Y | 1.4 | F262C | 1.4 | Q361T | 1.3 | A466Y | 1.3 |
| V120E | 1.3 | F262E | 1.6 | Q361V | 1.4 | D467I | 1.4 |
| E121I | 1.2 | F262H | 1.4 | Q361W | 1.5 | W469A | 1.4 |
| N125S | 1.4 | F262I | 1.4 | Q361Y | 1.6 | W469E | 1.4 |
| N126I | 1.2 | F262K | 1.8 | G362M | 1.4 | W469H | 1.3 |
| N128E | 1.5 | F262L | 1.4 | Y363A | 1.4 | W469L | 1.3 |
| Q129T | 1.2 | F262N | 1.6 | Y363E | 1.2 | W469N | 1.2 |
| I131Y | 1.3 | F262P | 2.0 | Y363F | 1.3 | W469Q | 1.3 |
| S132A | 1.3 | F262Q | 1.5 | Y363G | 1.7 | W469R | 1.3 |
| S132D | 1.4 | F262R | 1.7 | Y363I | 1.3 | W469S | 1.1 |
| S132H | 1.3 | F262S | 1.7 | Y363L | 1.2 | W469Y | 1.4 |
| S132I | 1.4 | F262T | 1.3 | Y363Q | 1.3 | N471A | 1.6 |
| S132K | 1.3 | F262V | 1.3 | Y363R | 1.1 | N471D | 1.5 |
| S132L | 1.4 | F262W | 1.5 | Y363S | 1.3 | N471M | 1.8 |
| S132P | 1.2 | A263E | 1.3 | Y363T | 1.4 | N471W | 1.2 |
| S132R | 1.4 | A263H | 1.4 | Y363V | 1.8 | S473N | 1.2 |
| S132T | 1.2 | A263W | 1.3 | Y363W | 1.7 | N475A | 1.5 |
| S132V | 1.3 | A265Q | 1.3 | P364C | 1.4 | N475C | 2.3 |
| S132Y | 1.3 | L272W | 1.2 | Y368C | 2.8 | N475D | 1.7 |
| G133E | 1.3 | G273Q | 1.2 | Y368Q | 1.4 | N475E | 1.5 |
| G133L | 1.2 | A274D | 1.1 | Y368R | 1.3 | N475F | 1.9 |
| G133T | 1.2 | L279I | 1.4 | Y368T | 1.2 | N475G | 1.6 |
| G133V | 1.2 | N280P | 1.2 | Y368Y | 1.4 | N475H | 1.7 |
| Y135C | 1.4 | K281H | 1.2 | Y372C | 1.7 | N475I | 1.3 |
| Y135D | 1.3 | K281I | 1.3 | Y372G | 1.4 | N475L | 1.9 |
| Y135E | 1.5 | K281S | 1.2 | Y372N | 1.2 | N475M | 1.4 |
| Y135P | 1.3 | N283P | 2.1 | Y372T | 1.2 | N475P | 1.6 |
| I137D | 1.2 | W284A | 1.3 | P375Q | 1.3 | N475Q | 1.6 |
| I137N | 1.3 | W284D | 1.5 | P375T | 1.3 | N475S | 1.4 |
| I137Q | 1.2 | W284E | 1.4 | P380F | 1.3 | N475T | 1.4 |
| I137W | 1.4 | W284G | 1.3 | M382I | 1.2 | N475V | 1.8 |
| I137Y | 1.2 | W284I | 1.4 | K383C | 1.3 | N475W | 1.5 |
| E138S | 1.4 | W284K | 1.4 | K383L | 1.2 | G476F | 1.2 |
| E138V | 1.4 | W284M | 1.4 | K383P | 1.2 | G476L | 1.3 |
| A139L | 1.7 | W284N | 1.4 | K383T | 1.7 | G477A | 2.3 |
| W140A | 1.5 | W284P | 1.5 | A384D | 1.4 | G477H | 2.1 |
| W140D | 1.4 | W284Q | 1.4 | A384N | 1.2 | G477Q | 1.8 |
| W140M | 1.5 | W284S | 1.5 | K385N | 1.3 | G477S | 1.7 |
| W140N | 1.4 | W284T | 1.3 | K385V | 1.3 | G477W | 1.4 |
| W140P | 1.4 | W284V | 1.5 | D387C | 1.4 | S478A | 1.4 |
| W140S | 1.4 | W284Y | 1.3 | D387P | 1.6 | S478F | 1.3 |
| W140T | 1.4 | S287I | 1.2 | D387R | 1.3 | S478Q | 1.2 |

The variants that showed an IF of at least 1.1 for Model A detergent are considered to have an improved performance when compared to the parent alpha-amylase, i.e. the alpha-amylase having the amino acid sequence set forth in SEQ ID NO: 1.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An alpha-amylase variant of a parent alpha-amylase having alpha-amylase activity comprising a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering; and wherein said variant has at least 60% but less than 100% sequence identity to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent polypeptide, preferably the polypeptide of SEQ ID NO: 1 and/or SEQ ID NO: 2.

2. The alpha-amylase variant according to claim 1, wherein said variant comprises one or more of the following substitutions at a position corresponding to positions N3A, N3C, N3D, N3E, N3F, N3G, N3H, N3L, N3P, N3Q, N3S, N3T, N3V, TSE, G7E, G7F, G7H, G7K, G7L, G7P, G7R, G7S, G7T, G7V, G7W, T8S, H16D, H16R, P18D, P18L, P18N, N25T, R26Y, D29N, D30F, R35P, R37A, R37N, T40A, T40C, T40D, T40E, T40G, T40H, T40I, T40K, T40N, T40V, T40W, A41C, A41D, A41E, A41H, A41I, A41K, A41N, A41R, A41T, A41V, A41Y, W43E, P46A, G50M, G50P, G50Q, G57A, G57S, A60E, A60S, A60V, E68F, E68Y, Q71F, G73D, G73H, G731, G73L, G73Q, G73S, G73T, G73W, T74F, K78S, K78T, T81M, Q841, Q84W, Q84Y, E86K, E86S, S87G, S87L, S87P, H90P, A91Q, A91V, A91W, K93V, V97C, V97H, Q98E, Q98F, Q98G, Q98I, Q98K, Q98L, Q98M, Q98R, Q98T, Q98Y, V103C, V103G, V103S, M105F, G110K, G110R, A111E, A111Q, A113T, T114M, T114V, N116C, N116Y, V117F, A119H, A119L, A119Y, V120E, E121I, N125S, N126I, N128E, Q129T, I131Y, S132A, S132D, S132H, S132I, S132K, S132L, S132P, S132R, S132T, S132V, S132Y, G133E, G133L, G133T, G133V, Y135C, Y135D, Y135E, Y135P, I137D, I137N, I137Q, I137W, I137Y, E138S, E138V, A139L, W140A, W140D, W140M, W140N, W140P, W140S, W140T, W140V, T141G, K142E, K142N, F143H, F143I, F143M, F145G, F145H, F145I, F145L, F145S, P146A, P146H, P146N, G147D, G149F, G149K, W159A, W159E, H161W, F162T, V165P, W167D, W167G, W167P, Q169L, Q169P, Q169V, Q169Y, R171P, R176E, R176Q, R176V, R176Y, I177H, I177P, K179A, K179M, K179V, F180D, F180G, R181D, R181E, R181G, R181M, R181Y, K185T, K185V, K185W, A186D, W187T, N195Y, G196D, N197V, N197Y, A204F, A204H, A204L, V206N, M208D, P211C, P211M, E216L, R218Q, R219V, T225D, T225F, T225H, T225K, T225L, T225N, T225R, T225Y, L228V, L230C, L230F, D231G, D231T, D231V, I235A, K242L, Y243D, Y243M, Y243N, R247G, R247P, R247S, L250S, L250Y, T251D, H252P, V253F, V253S, R254D, R254F, R254H, R254P, R254T, R254V, R254W, N255C, N255F, N255P, T257A, T257C, T257D, T257E, T257G, T257P, T257Q, T257S, T257W, T257Y, K259P, K259V, K259W, K259Y, E260V, M261D, M261F, M261P, M261R, M261S, M261W, F262A, F262C, F262E, F262H, F262I, F262K, F262L, F262N, F262P, F262Q, F262R, F262S, F262T, F262V, F262W, A263E, A263H, A263W, A265Q, L272W, G273Q, A274D, L279I, N280P, K281H, K281I, K281S, N283P, W284A, W284D, W284E, W284G, W284I, W284K, W284M, W284N, W284P, W284Q, W284S, W284T, W284V, W284Y, S287I, S287K, S287L, S287P, S287R, S287T, S287V, S287Y, P292L, P292M, P292Y, H294N, H294S, N296T, Y298D, Y298E, N299T, A300G, S303A, S303C, S303D, S303E, S303F, S303G, S303H, S303L, S303M, S303N, S303Q, S303T, S303V, S303Y, G305E, Y307S, A310D, A310N, K311A, K311C, L313Q, N314P, N314S, G315E, G315M, G315V, Q319C, Q319E, K320S, K320W, H321F, H321K, H321L, H321R, H321T, H321V, H321W, P322C, P322E, P322H, P322T, P322Y, M323A, M323C, M323D, M323E, M323G, M323H, M323L, M323N, M323P, M323R, M323S, M323T, M323W, M323Y, H324A, H324I, H324P, H324T, H324W, H324Y, S334V, G337V, L340M, S342A, S342G, S342T, V344A, V344C, V344I, Q345N, E346A, W347A, W347E, W347F, W347G, W347H, W347K, W347N, W347P, W347R, P350A, P350C, P350N, P350T, R359C, R359F, R359G, R359M, R359S, R359W, E360D, E360H, E360P, E360T, E360W, Q361D, Q361H, Q361I, Q361K, Q361L, Q361M, Q361N, Q361P, Q361R, Q361S, Q361T, Q361V, Q361W, Q361Y, G362M, Y363A, Y363E, Y363F, Y363G, Y363I, Y363L, Y363Q, Y363R, Y363S, Y363T, Y363V, Y363W, P364C, Y368C, Y368Q, Y368R, Y368T, Y372C, Y372G, Y372N, Y372T, P375Q, P375T, P380F, M382I, K383C, K383L, K383P, K383T, A384D, A384N, K385N, K385V, D387C, D387P, D387R, D387W, P388I, E391M, R393K, R393M, N395C, N395D, N395K, N395V, N395W, F396E, F396G, F396T, A397V, Y398E, Q401H, H402S, D403A, D403E, D403T, F405C, F405N, F405S, F405T, D406V, H407C, H407Q, H407T, I41C H, I41C M, I41C R, I41C W, R415C, R415L, R415M, R415Y, E416D, E416F, E416L, E416N, N418A, N418P, N418V, H421A, H421G, H421L, H421P, H421Q, H421Y, P422D, P422G, P422I, P422N, P422Q, P422S, N423G, N423H, N423I, N423L, N423M, N423P, N423Q, N423V, N423W, N423Y, G433A, G433N, G433S, P434I, P434L, G435C, G435L, G435M, G435T, G435V, K438A, K438I, K438Q, K438S, K438V, W439A, W439D, W439I, W439K, W439L, W439N, W439S, W439T, W439V, W439Y, K446A, K446F, A447D, A447Y, Q449T, V450D, V450N, H452L, T455C, T455M, T455V, N457D, N457E, N457I, N457L, N457V, K458D, K458L, G460M, G460V, T461F, T461I, T461Q, T461V, V462C, N465A, N465C, N465E, N465G, N465H, N465L, N465P, N465T, A466F, A466I, A466Y, D467I, W469A, W469E, W469H, W469L, W469N, W469Q, W469R, W469S, W469Y, N471A, N471D, N471M, N471W, S473N, N475A, N475C, N475D, N475E, N475F, N475G, N475H, N475I, N475L, N475M, N475P, N475Q, N475S, N475T, N475V, N475W, G476F, G476L, G477A, G477H, G477Q, G477S, G477W, S478A, S478F, and S478Q; using SEQ ID NO: 1 for numbering and wherein said variant has at least 60% but less than 100% sequence identity to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent polypeptide, preferably the polypeptide of SEQ ID NO: 1 and/or SEQ ID NO: 2.

3. The alpha-amylase variant according to claim 1, wherein said improved property relative to said parent polypeptide of SEQ ID NO: 1 is increased specific activity, particularly increased specific activity in Model A detergent composition.

4. The alpha-amylase variant according to claim 1, wherein said improved property is increased specific activity measured as an Improvement Factor (IF) of >1.0 when compared to said parent polypeptide of SEQ ID NO: 1 having alpha-amylase activity.

5. The alpha-amylase variant according to claim 1, further comprising deletion at two or more positions corresponding to positions R181, G182, D183, and G184, preferably D183* and G184*, using SEQ ID NO: 1 for numbering.

6. The alpha-amylase variant according to claim 1, wherein the parent polypeptide has at least 60%, but less than 100% sequence identity to the polypeptide of SEQ ID NO: 1, 2, 3, 4, 5, 6, 7, 8, 9, 10, 11, 12, 13, 14, 15, 16 or 17.

7. The alpha-amylase variant according to claim 1, wherein the variant has at least 60%, but less than 100% sequence identity to the amino acid sequence of the parent polypeptide.

8. A polynucleotide encoding the variant according to claim 1.

9. A nucleic acid construct or an expression vector comprising the polynucleotide according to claim 8.

10. A host cell comprising, the nucleic acid construct or the expression vector according to claim 9.

11. A composition comprising the variant according to claim 1 and at least one additional active component.

12. The composition according to claim 11, wherein said composition is a detergent composition, such as a liquid or powder detergent composition.

13. The composition according to claim 11, wherein said composition is a liquid laundry or liquid dish wash composition, such as an Automatic Dish Wash (ADW) liquid detergent composition, or a powder laundry, such as a soap bar, or powder dish wash composition, such as an ADW unit dose detergent composition and such as a Hand Dish Wash (HDW) detergent composition.

14. A method of obtaining an alpha-amylase variant of a parent alpha-amylase comprising the steps of:
a) introducing a substitution at one or more positions corresponding to positions: 3, 5, 7, 8, 16, 18, 25, 26, 29, 30, 35, 37, 40, 41, 43, 46, 50, 57, 60, 68, 71, 73, 74, 78, 81, 84, 86, 87, 90, 91, 93, 97, 98, 103, 105, 110, 111, 113, 114, 116, 117, 119, 120, 121, 125, 126, 128, 129, 131, 132, 133, 135, 137, 138, 139, 140, 141, 142,143, 145, 146, 147, 149, 159, 161, 162, 165, 167, 169, 171, 176, 177, 179, 180, 181, 185, 186, 187, 195, 196, 197, 204, 206, 208, 211, 216, 218, 219, 225, 228, 230, 231, 235, 242, 243, 247, 250, 251, 252, 253, 254, 255, 257, 259, 260, 261, 262, 263, 265, 272, 273, 274, 279, 280, 281, 283, 284, 287, 292, 294, 296, 298, 299, 300, 303, 305, 307, 310, 311, 313, 314, 315, 319, 320, 321, 322, 323, 324, 334, 337, 339, 340, 342, 344, 345, 346, 347, 350, 359, 360, 361, 362, 363, 364, 368, 372, 375, 380, 382, 383, 384, 385, 387, 388, 391, 393, 395, 396, 397, 398, 401, 402, 403, 405, 406, 407, 410, 415, 416, 418, 421, 422, 423, 433, 434, 435, 438, 439, 446, 447, 449, 450, 452, 455, 457, 458, 460, 461, 462, 465, 466, 467, 469, 471, 473, 475, 476, 477, and 478, using SEQ ID NO: 1 for numbering;
b) optionally introducing a deletion at two or more positions in the parent alpha-amylase said positions R181, G182, D183, and G184, using SEQ ID NO: 1 for numbering;
and said method thereby providing an alpha-amylase variant of said parent alpha-amylase, wherein said variant has at least 60% but less than 100%, sequence identity to the amino acid sequence to the polypeptide of SEQ ID NOs: 1, 2, 3, 4, 5, 6, 7, 8, 9, 10, 11, 12, 13, 14, 15, 16 or 17, and wherein said variant has alpha-amylase activity and wherein the alpha-amylase variant has an improved property relative to said parent.

15. (canceled)
